(19) United States
(12) Patent Application Publication
(10) Pub. No.: US 2024/0269452 A1
(43) Pub. Date: Aug. 15, 2024

(54) MEDICAL CONNECTORS

(71) Applicant: ICU Medical, Inc., San Clemente, CA (US)

(72) Inventors: Trent Spencer Wells, Santa Ana, CA (US); David Nelson, Lake Forest, CA (US); Erik Scott Shauver, Tustin, CA (US)

(21) Appl. No.: 18/511,776

(22) Filed: Nov. 16, 2023

Related U.S. Application Data
(60) Provisional application No. 63/426,314, filed on Nov. 17, 2022.

(51) Int. Cl. A61M 39/10; A61M 39/22

(52) U.S. Cl. CPC A61M 39/1011; A61M 39/22; A61M 2039/1077

(57) ABSTRACT

A medical connector can include a threaded connection fitting configured to couple to a medical implement by rotation of the medical implement in a tightening direction relative to the threaded connection fitting. The threaded connection fitting can include one or more first engagement features. The connector can have a fluid pathway for transferring fluid through the device and a rotation mechanism. The rotation mechanism permits the threaded connection fitting to rotate in a loosening direction relative to the housing, such as to impede unintended disconnection of the medical implement from the threaded connection fitting. In some cases, the connector can have a disengaged configuration that permits the threaded connection fitting to rotate relative to the housing in the tightening direction and in the loosening direction.

### CROSS-REFERENCE TO RELATED APPLICATIONS

This application claims the benefit under 35 U.S.C. § 119(e) of U.S. Provisional Patent Application No. 63/426,314, filed Nov. 17, 2022, and titled MEDICAL CONNECTORS. The entirety contents of each of the above-identified application(s) are hereby incorporated by reference herein and made part of this specification for all that they disclose.

### BACKGROUND

### Field of the Disclosure

Some embodiments disclosed herein relate to medical connector.

### Description of the Related Art

Although various medical connectors exist there remains a need for improved medical connectors.

### SUMMARY OF CERTAIN INVENTIVE ASPECTS

Without limiting the scope of the claims, certain example embodiments are summarized below for illustrative purposes. Embodiments may include several novel features, no single one of which is solely responsible for its desirable attributes or which is essential to the embodiments.

In accordance with some aspects of the disclosure, a medical connector can include a housing that has first, second, third, and fourth sidewall sections. The second and fourth sidewall sections can have protrusions that extend inwardly. The second and fourth sidewall sections can be configured to be pressed inwardly towards each other to transition the medical connector from a disengaged state to an engaged state when an external force is applied. The second and fourth sidewall sections can be configured to automatically move outwardly away from each other to transition the medical connector from the engaged state to the disengaged state when the external force is removed. The connector can have a proximal end portion that can have a connection fitting configured to couple to another connector by rotation of the other connector relative to the proximal end portion in a tightening direction. The proximal end portion can include protrusions configured to engage the protrusions on the second and fourth sidewall sections of the housing when the medical connector is in the engaged state to impede rotation of the proximal end portion relative to the housing in the tightening direction to facilitate coupling of the other connector to the proximal end portion. The protrusions on the proximal end portion can be configured to not engage the protrusions on the second and fourth sidewall sections of the housing when the medical connector is in the disengaged state, so that the proximal end portion rotates relative to the housing in the tightening direction and in a loosening direction opposite the tightening direction.

In accordance with some aspects of the disclosure, a medical connector can include a first portion (e.g., first housing portion) with a first opening, a second portion (e.g., second housing portion) with a second opening, a fluid pathway extending between the first opening and the second opening, and a rotation mechanism having a first configuration and a second configuration. The first configuration can permit rotation of the first portion relative to the second portion in a first direction and in a second direction. The second configuration can impede rotation of the first portion relative to the second portion in the second direction. The rotation mechanism can be in the first configuration by default.

In accordance with some aspects of the disclosure, a medical fluid transfer device can include a threaded connection fitting configured to couple to a first medical implement by rotation of the first medical implement in a tightening direction relative to the threaded connection fitting. The threaded connection fitting can include one or more first engagement features. The device can have a fluid pathway for transferring fluid through the device. The device can have a housing that includes at least one movable section with one or more second engagement features. The movable section can have an engaged configuration and a disengaged configuration. The engaged configuration can have the one or more second engagement features positioned to engage with the one or more first engagement features to impede the threaded connection fitting from rotating in the tightening direction relative to the housing to facilitate coupling of the first medical implement to the threaded connection fitting. The engaged configuration can permit the threaded connection fitting to rotate in a loosening direction relative to the housing to impede unintended disconnection of the first medical implement from the threaded connection fitting. The disengaged configuration can position the second engagement features to be disengaged from the first engagement features so that the threaded connection fitting can rotate relative to the housing in the tightening direction and in the loosening direction.

In accordance with some aspects of the disclosure, a medical connector can include a first connector portion that includes a first opening and one or more first engagement features, a second connector portion that includes a second opening and one or more second engagement features, a fluid pathway between the first opening and the second opening and configured for transferring fluid through the medical connector, and an engagement member that has one or more inner engagement features and one or more outer engagement features. The one or more inner engagement features can be configured to interface with the one or more first engagement features on the first connector portion. The one or more outer engagement features can be configured to interface with the one or more second engagement features on the second connector portion. The medical connector can permit rotation of the first connector portion relative to the second connector portion in a first direction while impeding rotation of the first connector portion relative to the second connector portion in a second direction that is opposite the first direction.

In accordance with some aspects of the disclosure, a medical connector can include a proximal portion with a proximal opening, a distal portion with a distal opening, a fluid pathway extending between the proximal opening and the distal opening, and a rotation mechanism that permits rotation of the proximal portion relative to the distal portion. The medical connector can include any of the various features disclosed herein.

In accordance with some aspects of the disclosure, a medical connector can include a housing comprising first, second, third, and fourth sidewall sections, wherein the second and fourth sidewall sections can include protrusions that extend inwardly, wherein the second and fourth sidewall sections can be configured to be pressed inwardly towards each other to transition the medical connector from a disengaged state to an engaged state when an external force is applied, and wherein the second and fourth sidewall section are configured to automatically move outwardly away from each other to transition the medical connector from the engaged state to the disengaged state when the external force is removed. A proximal end portion can include a connection fitting configured to couple to another connector by rotation of the other connector relative to the proximal end portion in a tightening direction. The proximal end portion can include protrusions configured to engage the protrusions on the second and fourth sidewall sections of the housing when the medical connector is in the engaged state to impede rotation of the proximal end portion relative to the housing in the tightening direction to facilitate coupling of the other connector to the proximal end portion. The protrusions on the proximal end portion can be configured to not engage the protrusions on the second and fourth sidewall sections of the housing when the medical connector is in the disengaged state, so that the proximal end portion rotates relative to the housing in the tightening direction and in a loosening direction opposite the tightening direction.

The medical connector can be configured to permit rotation of the proximal end portion relative to the housing in the loosening direction when the medical connector is in the engaged state, such as to impede disconnection of the other connector from the proximal end portion. The protrusions on the proximal end portion and/or the protrusions on the housing can be configured to deform to permit the proximal end portion to rotate relative to the housing in the loosening direction when the medical connector is in the engaged state. Either the protrusions on the proximal end portion and/or the protrusions on the housing can include pawls that are configured to flex to permit the protrusions on the proximal end portion to rotate past the protrusions on the distal end portion in the loosening direction when the medical connector is in the engaged state. The medical connector can be configured such that when the medical connector is in the engaged state, rotating the proximal end portion relative to the housing in the loosening direction by a force that is below a disconnection threshold for loosening or removing the attached other connector from the proximal end portion causes the protrusions on the proximal end portion to rotate past the protrusions on the housing, such as to thereby impede disconnection of the other connector from the proximal end portion. The protrusions on the proximal end portion can include flexible pawls. The protrusions on the second the fourth sidewall sections of the housing can include flexible pawls.

The first and third sidewall sections can define stop surfaces that limit the distance that the second and fourth sidewall sections can be pressed inwardly towards each other. The first and third sidewall sections can be positioned to abut against the second and fourth sidewall sections when the medical connector is in the engaged state, such as to limit engagement between the protrusions so that the proximal end portion can rotate in the loosening direction relative to the housing. The connection fitting on the proximal end portion can be a luer lock fitting. The connection fitting on the proximal end portion can be a female luer lock fitting. The connection fitting on the proximal end portion can be a female luer. The connection fitting on the distal end portion can be a male luer (e.g., a male luer lock fitting). In some implementations, the proximal end portion does not move axially relative to the housing. Gaps can separate the first, second, third, and fourth sidewall sections. The protrusions on the housing can be configured to directly contact the protrusions on the proximal end portion in the engaged state.

The medical connector can include an engagement member that has one or more inner engagement elements that are configured to interface with the protrusions on the proximal end portion, and the engagement member can have one or more outer engagement elements that are configured to interface with the protrusions on the housing. The proximal end portion can include a first opening, the housing can include a second opening, and the medical connector can include a fluid pathway between the first opening and the second opening. The medical connector can include a valve member configured to move between a closed position that closes the fluid pathway and an open position that opens the fluid pathway. The valve member can include a fluid channel. A proximal end of the valve member can extend into the proximal end portion. The medical connector can include a seal member configured to provide a fluid seal between an exterior of the valve member and an interior of the proximal end portion. The medical connector, can include a biasing member configured to bias the valve member toward the cloased position. The biasing member can include a first securing portion that can be coupled to the housing, a second securing portion that can be coupled to the valve member, and one or more resilient straps that can couple the first securing portion to the second securing portion. The second securing portion can move away from the first securing portion as the valve member moves from the closed position toward the open position, thereby stretching the one or more resilient straps. The biasing member can be a spring.

In accordance with some aspects of the disclosure, a medical connector can include a first portion with a first opening; a second portion with a second opening; a fluid pathway extending between the first opening and the second opening; and a rotation mechanism having a first configuration and a second configuration. The first configuration can permit rotation of the second portion relative to the first portion in a first direction and in a second direction that is opposite the first direction. The second configuration can impede rotation of the second portion relative to the first portion in the second direction.

The rotation mechanism can be in the first configuration by default. The second configuration of the rotation mechanism can permit rotation of the second portion relative to the first portion in the first direction and can impede rotation of the second portion relative to the first portion in the second direction. The second portion can be configured to couple to a medical implement by rotation of the medical implement in the second direction (e.g., for threaded engagement between the medical implement and the second portion). The rotation mechanism can be configured to transition between the first configuration and the second configuration without axial movement of the second portion relative to the first portion. The rotation mechanism can include one or more engagement elements on the first portion that are configured to engage engagement elements on the second portion when the rotation mechanism is in the second configuration, for example so that the second portion is impeded from rotating relative to the first portion in the second direction. The one or more engagement elements on the first portion or the one or more engagement elements on the second portion can be configured to flex to permit the one or more engagement elements on the second portion to rotate in the first direction past the one or more engagement elements on the first portion when the rotation mechanism is in the second configuration. The one or more engagement elements on the first portion can be configured to directly contact the one or more engagement elements on the second portion when the rotation mechanism is in the second configuration. The one or more engagement elements on the second portion can be configured to rotate without contacting the one or more engagement elements on the first portion when the rotation mechanism is in the first configuration.

The rotation mechanism can include an engagement member that can have one or more inner engagement elements that are configured to interface with the one or more engagement elements on the second portion, and the engagement member can have one or more outer engagement elements that are configured to interface with the one or more engagement elements on the first portion. The engagement member can include a body portion that has a substantially circular shape. The engagement member can be configured to be deformed away from the substantially circular shape when the rotation mechanism is in the second configuration.

The first portion can include one or more movable sections that can be actuated by a user to move the one or more engagement elements on the first portion towards the one or more engagement elements on the second portion, such as to transition the rotation mechanism to the second configuration. The first portion can include at least one stop to limit the distance that the one or more movable sections can be actuated. The second configuration of the rotation mechanism can impede rotation of the second portion relative to the first portion in the first direction and in the second direction.

In some implementations, the second portion can move axially relative to the first portion to transition the rotation mechanism between the first configuration and the second configuration. A biasing member can be configured to bias the first portion toward an axial position that produces the first configuration of the rotation mechanism. The first portion can include one or more protrusions or recesses that engage one or more corresponding recesses or protrusions on the second portion when the rotation mechanism is in the second configuration. The one or protrusions or recesses can be disengaged from the one or more corresponding recesses or protrusions when the rotation mechanism is in the first configuration.

The medical connector can include a valve having a closed configuration that closes the fluid pathway and an open configuration that opens the fluid pathway. The valve member can include a fluid channel. The connector can include a first seal member, which can be configured to provide a fluid seal between an exterior of the valve member and the first portion of the connector. The connector can include a second seal member, which can be configured to provide a fluid seal between an exterior of the valve member and the second portion of the connector. A biasing member can be configured to bias the valve to the cloased configuration. The biasing member can include a first securing portion coupled to the first portion of the connector, a second securing portion coupled to the valve, and one or more resilient straps that couple the first securing portion to the second securing portion.

The first portion can be configured to connect to a first medical implement, and the second portion can be configured to connect to a second medical implement. The medical connector can be configured to transfer fluid between the first medical implement and the second medical implement. The second portion can include a luer lock fitting. The first portion can include a luer lock fitting. The second portion can include a female luer lock fitting, and the first portion can include a male luer lock fitting.

In accordance with some aspects of the disclosure, a medical fluid transfer device can include a threaded connection fitting that can be configured to couple to a medical implement by rotation of the medical implement in a tightening direction relative to the threaded connection fitting. The threaded connection fitting can include one or more first engagement features. The medical connector can include a fluid pathway for transferring fluid through the device. The medical connector can include a housing that can include at least one movable section with one or more second engagement features, and the movable section can have an engaged configuration and a disengaged configuration. The engaged configuration can have the one or more second engagement features positioned to engage with the one or more first engagement features to impede the threaded connection fitting from rotating in the tightening direction relative to the housing, such as to facilitate coupling of the medical implement to the threaded connection fitting. The engaged configuration can permit the threaded connection fitting to rotate in a loosening direction relative to the housing, such as to impede unintended disconnection of the medical implement from the threaded connection fitting. The disengaged configuration can position the second engagement features to be disengaged from the first engagement features so that the threaded connection fitting can rotate relative to the housing in the tightening direction and in the loosening direction.

The one or more first engagement features or the one or more second engagement features can be configured to flex to permit the first engagement features to rotate past the second engagement features in the loosening direction. The one or more first engagement features or the one or more second engagement features can be configured to flex radially. The threaded connection fitting can be locked against axial movement relative to the housing. The movable section of the housing can be configured to move radially to transition between the engaged configuration and the disengaged configuration. The movable section of the housing can be biased toward the disengaged configuration. A stop can limit the distance that the movable section of the housing can be moved. The at least one movable section can include two opposing sidewall sections configured to be pinched together to transition to the engaged configuration.

The medical connector can include a valve member, which can be configured to move between a closed position that closes the fluid pathway and an open position that opens the fluid pathway. The valve member can include a fluid channel with an opening at an end of the valve member. The end of the valve member can be disposed in inside of the threaded connection fitting. A seal member can be configured to provide a fluid seal between an exterior of the valve member and an interior of the threaded connection fitting. The medical connector can include a biasing member to bias the valve member toward the closed position. The biasing member can include a first securing portion coupled to the housing, a second securing portion coupled to the valve member, and one or more resilient portions or straps that couple the first securing portion to the second securing portion. The second securing portion can move away from the first securing portion as the valve member moves from the closed position toward the open position, thereby stretching the one or more resilient straps.

In accordance with some aspects of the disclosure, a medical connector can include a firs housing portion with a first opening, a second housing portion with a second opening, a fluid pathway extending between the first opening and the second opening, and a rotation mechanism that permits rotation of the second housing portion relative to the first housing portion. The medical connector can include any of the features disclosed herein.

In accordance with some aspects of the disclosure, a medical connector can include a first housing portion having a hollow projection. The first housing portion can an interior volume. The medical connector can include a second housing portion, which can be partially disposed in the interior volume of the first housing portion, the second housing portion coupled to the first housing portion so as to permit the second housing portion to rotate relative to the first housing portion in at least a first direction. The connector can include a valve member that includes a fluid channel having a first end that is disposed inside the hollow projection and a second end that is disposed inside the second housing portion. The valve member can be movable between a closed configuration and an open configuration. The connector can include a biasing member configured to bias the valve member toward the closed configuration.

The first housing portion can include a male luer. The second housing portion can include a female luer. The second housing portion can include a syringe body portion, vial adapter body portion, or a bag spike body portion. The medical connector can include a seal member configured to provide a fluid seal between an exterior of the valve member and an interior of the second housing portion. The connector can include a seal member configured to provide a fluid seal between an exterior of the valve member and an interior of the first housing portion. The medical connector can be configured to permit the second housing portion to rotate relative to the first housing portion in a second direction that is opposite the first direction. The medical connector can be configured to impede rotation of the second housing portion relative to the first housing portion in a second direction that is opposite the first direction. The connector can have a first configuration in which the second housing portion is permitted to rotate relative to the first housing portion in both the first direction and in a second direction that is opposite the first direction, and the connector can have a second configuration in which the second housing portion is permitted to rotate relative to the first housing portion in the first direction and where the second housig portion is impeded from rotating relative to the first housing portion in the second direction. The second housing portion can be configured to couple to a medical implement by rotation of the medical implement in the second direction. The medical connector can include a biasing member configured to bias the valve member toward the closed position. The biasing member can include a first securing portion coupled to the housing, a second securing portion coupled to the valve member, and one or more resilient straps that couple the first securing portion to the second securing portion, wherein the second securing portion moves away from the first securing portion as the valve member moves from the closed position toward the open position, thereby stretching the one or more resilient straps. The second housing portion can extend longitudinally out of the first housing portion by a distance between about 3 mm and about 9 mm, in some implementations. A first part of the second housing portion can be received into the first housing portion, and a second part of the second housing portion can extend out of the first housing portion. The second part of the second housing portion can include a female luer lock with external threading. In some cases, the second housing portion does not have any protrusions other than the external threading of the female luer lock. When a standard male lure lock connector is coupled to the second housing portion, the standard male lure lock connector can be spaced away from the first housing portion by a gap of less than 7 mm, such as a gap between about 1 mm and about 3 mm. A structure to impede rotation of the second housing portion relative to the first housing portion (e.g., one or more pawls, protrusions, or teeth) is disposed inside of the first housing portion.

In accordance with some aspects of the disclosure, a medical connector can include a first connector portion that includes a first opening and one or more first engagement features, a second connector portion that includes a second opening and one or more second engagement features, a fluid pathway between the first opening and the second opening and configured for transferring fluid through the medical connector, and an engagement member that has one or more inner engagement features and one or more outer engagement features. The one or more inner engagement features can be configured to interface with the one or more first engagement features on the first connector portion. The one or more outer engagement features can be configured to interface with the one or more second engagement features on the second connector portion. The medical connector can permit rotation of the first connector portion relative to the second connector portion in a first direction while impeding rotation of the first connector portion relative to the second connector portion in a second direction that is opposite the first direction.

The engagement member can be keyed with a portion of the first connector portion, for example so that the engagement member rotates with the first connector portion in the first direction and/or the second direction. The engagement member can be configured to rotate relative to the first connector portion until the one or more first engagement features on the first connector portion engage the one or more inner engagement features on the engagement member. The engagement member can be configured to rotate relative to the second connector portion until the one or more second engagement features on the second connector portion engage the one or more outer engagement features on the engagement member.

### BRIEF DESCRIPTION OF THE DRAWINGS

Embodiments of this disclosure will now be described, by way of non-limiting example, with reference to the accompanying drawings.

FIG. 1 is a schematic diagram showing an example embodiment of a medical connector.

FIG. 2 shows a cross-sectional view of an example embodiment of a medical connector.

FIG. 3 is a partial view of an example embodiment of a connector with a one-way rotation mechanism.

FIG. 4 is a partial exploded view of the connector of FIG. 3.

FIG. 5 is a partial cross-sectional view of the connector of FIG. 3.

FIG. 6 shows an example embodiment of an engagement member.

FIG. 7 is a partial perspective view of an example embodiment of a connector.

FIG. 8 is a partial exploded view of the example connector of FIG. 7.

FIG. 9 is a partial cross-sectional view of the connector of FIG. 7.

FIG. 10 is a cross-sectional view through the rotation mechanism of the connector of FIG. 7, showing rotation in a first direction.

FIG. 11 is a cross-sectional view through the rotation mechanism of the connector of FIG. 7, showing rotation in a second direction.

FIG. 12 is a detailed view of part of the cross-sectional view of FIG. 11.

FIG. 13 is a partial exploded view of another example embodiment of a connector.

FIG. 14 is a partial cross-sectional view of the example connector of FIG. 13.

FIG. 15 is a cross-sectional view taken through the rotation mechanism of the example connector of FIG. 13.

FIG. 16 shows a partial exploded view of an example embodiment of a connector.

FIG. 17 shows a partial cross-sectional view of the connector of FIG. 16.

FIG. 18 is a partial exploded view of another example embodiment of a connector.

FIG. 19 is a partial cross-sectional view of the example connector of FIG. 18.

FIG. 20 shows a partial view of components of the example connector of FIG. 18.

FIG. 21 shows a partial view of components of the example connector of FIG. 18.

FIG. 22 shows a partial exploded view of another example embodiment of a connector.

FIG. 23 shows a partial perspective view of the example connector of FIG. 22 in a free-spin configuration.

FIG. 24 shows a partial perspective view of the example connector of FIG. 22 in an engaged configuration.

FIG. 25 shows a partial cross-sectional view of the example connector of FIG. 22 in a free-spin configuration.

FIG. 26 shows a partial cross-sectional view of the example connector of FIG. 22 in an engaged configuration.

FIG. 27 shows a partial exploded view of another example embodiment of a connector.

FIG. 28 shows a partial perspective view of the example connector of FIG. 27.

FIG. 29 shows a partial cross-sectional view of the example connector of FIG. 27 in a free-spin or disengaged configuration.

FIG. 30 shows a partial cross-sectional view of the example connector of FIG. 27 in an engaged configuration.

FIG. 31 shows a cross-sectional view through the rotation mechanism of the example connector of FIG. 27 in a free-spin configuration.

FIG. 32 shows a cross-sectional view through the rotation mechanism of the example connector of FIG. 27 in an engaged configuration.

FIG. 33 shows a partial cross-sectional view of the example connector.

FIG. 34 shows a cross-sectional view through the rotation mechanism of the example connector of FIG. 33 in a free-spin or disengaged configuration.

FIG. 35 shows a cross-sectional view through the rotation mechanism of the example connector of FIG. 33 in an engaged configuration.

FIG. 36 shows a partial exploded view of another example embodiment of a connector.

FIG. 37 shows a partial cross-sectional view of the example connector of FIG. 36.

FIG. 38 shows a cross-sectional view through the rotation mechanism of the example connector of FIG. 36.

FIG. 39 shows another cross-sectional view through the rotation mechanism of the example connector of FIG. 36.

FIG. 40 shows a perspective view of another example embodiment of a connector.

FIG. 41 shows proximal housing portion of the example connector of FIG. 40.

FIG. 42 shows a cross-sectional view of the example connector of FIG. 40.

FIG. 43 shows another cross-sectional view of the example connector of FIG. 40.

FIG. 44 shows a cross-sectional view of another example embodiment of a connector 2002.

FIG. 45 is a partial perspective view of the example connector 2002 of FIG. 44.

FIG. 46 is a partial perspective view of the example connector 2002 of FIG. 44 with a portion of the housing cut away to facilitate illustration of the interior of the connector.

FIG. 47 shows an example embodiment of a housing for a connector.

FIG. 48 shows an example embodiment of a housing for a connector.

FIG. 49 shows an example embodiment of a housing for a connector.

FIG. 50 shows a cross-sectional view of an example connector in a disengaged configuration.

FIG. 51 shows a cross-sectional view of the example connector of FIG. 50 in an engaged configuration.

FIG. 52 shows a cross-sectional view of the example connector of FIG. 50 in an engaged configuration.

FIG. 53 shows a cross-sectional view of the example connector of FIG. 50 in an additional configuration.

FIG. 54 shows a cross-sectional view of the example connector of FIG. 50 in an additional configuration.

FIG. 55 is a perspective view of an example embodiment of a connector.

FIG. 56 is another perspective view of the example connector of FIG. 55.

FIG. 57 is an exploded view of the example connector of FIG. 55.

FIG. 58 is another exploded view of the example connector of FIG. 55.

FIG. 59 is a cross-sectional view of the example connector.

FIG. 60 is another cross-sectional view of the example connector.

FIG. 61 is another cross-sectional view of the example connector.

FIG. 62 shows a perspective view of the component of the example connector with the actuation arms.

FIG. 63 shows a cross-sectional view of an example embodiment of a connector.

FIG. 64 shows another cross-sectional view of the example embodiment of a connector.

FIG. 65 is a cross-sectional taken through a rotation mechanism of an example embodiment of a connector in a disengaged configuration.

FIG. 66 is a cross-sectional taken through the rotation mechanism of the example embodiment of a connector in an engaged configuration.

FIG. 67 is a cross-sectional taken through a rotation mechanism of another example embodiment of a connector.

FIG. 68 shows a cross-sectional view of an example embodiment of a connector.

FIG. 69 shows another cross-sectional view of the example embodiment of a connector.

FIG. 70 shows a cross-sectional view of another example embodiment of a connector.

FIG. 71 shows another cross-sectional view of the example embodiment of a connector.

FIG. 72 shows an example implementation of a connector coupled to a body of a syring.

FIG. 73 shows another example connector coupled to a body of a syring.

### DETAILED DESCRIPTION OF CERTAIN EMBODIMENTS

The various features and advantages of the systems, devices, and methods of the technology described herein will become more fully apparent from the following description of the examples illustrated in the figures. These examples are intended to illustrate the principles of this disclosure, and this disclosure should not be limited to merely the illustrated examples. The features of the illustrated examples can be modified, combined, removed, and/or substituted as will be apparent to those of ordinary skill in the art upon consideration of the principles disclosed herein.

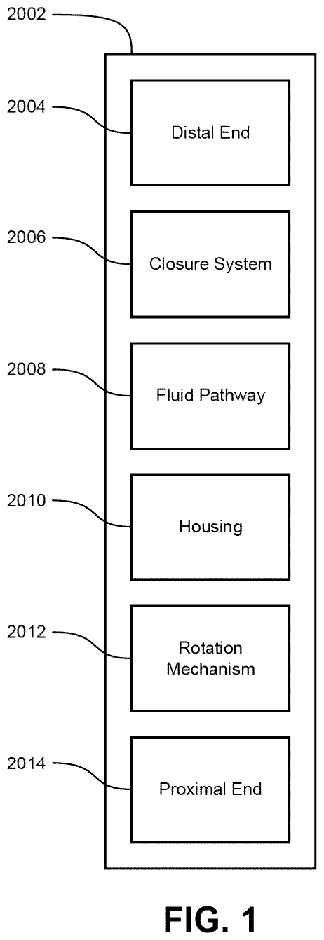

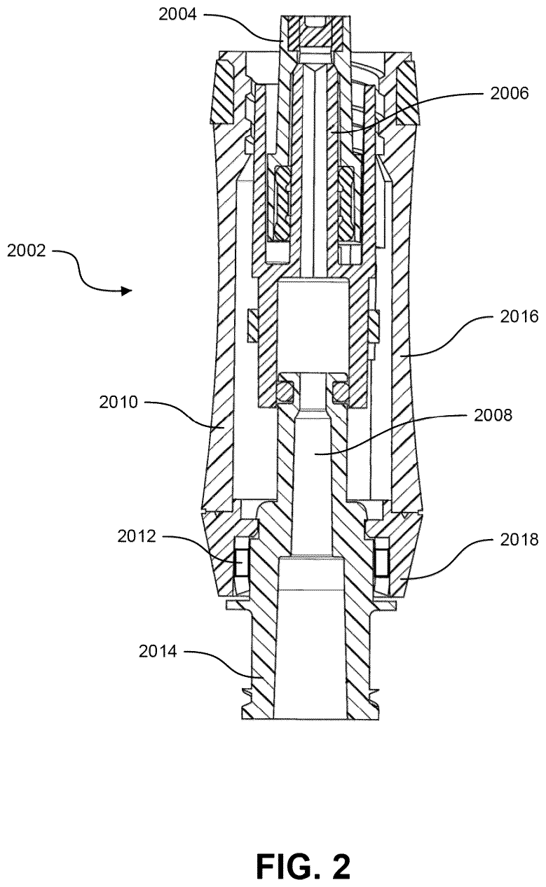

FIG. 1 is a schematic diagram showing an example embodiment of a medical connector 2002. The medical connector 2002 can have a distal end 2004, a closure system 2006, a fluid pathway 2008, a housing 2010, a rotation mechanism 2012, and a proximal end 2014. FIG. 2 shows a cross-sectional view of an example embodiment of a medical connector 2002. By way of example, the medical connector 2002 have features similar to the connector embodiments disclosed in U.S. Patent Application Publication No. 2019/0078712 (the “′712 Publication”), published Mar. 14, 2019, and titled AXIALLY ENGAGING MEDICAL CONNECTOR SYSTEM WITH DIMINISHED FLUID REMNANTS, which is hereby incorporated by reference in its entirety.

The connector 2002 can have a distal end 2004, which can include a male luer (e.g., similar to the male luer tip 122 of the ′712 Publication). The male luer can be surrounded by a shroud, which can have internal threading, such as to engage threading or protrusion(s) on a corresponding female luer. The shroud can be part of the housing 2010. In some embodiments, the distal end 2004 can be integral with, or functionally unitary with (e.g., separately formed but joined to move together), the housing 2010. The distal end 2004 can have a distal opening, such as at the end of the male luer.

The connector 2002 can have a proximal end 2014, which can be a female luer. The proximal end 2014 can be similar to the second end 112 or first cap component 132 of the ′712 Publication. The proximal end 2014 can have external threading or protrusions, such as to engage a locking male luer of another connector. The proximal end 2014 can have a proximal opening, such as at the end of the female luer.

A fluid pathway 2008 can extend between the distal end 2004 and the proximal end 2014 of the connector 2002, such as to enable fluid to be transferred through the connector 2002. In some embodiments, the fluid pathway within the connector 2002 may be continuous or without interruption between the distal end 2004 and proximal end 2014. The connector 2002 can have a closure system 2006, which can include a valve or seal. The closure system 2006 can have an open configuration, which can enable fluid to flow through the fluid pathway 2008, such as between the distal end 2004 and the proximal end 2014. The closure system 2006 can have a closed configuration, which can impede fluid from flowing through the fluid pathway 2008. For example, the closure system 2006 can close the distal end 2004 of the connector 2002, in some embodiments, although any suitable closure system can be used. In some embodiments, the closure system 2006 can automatically transition to the open configuration when a corresponding connector is attached to the connector 2002 (e.g., to the distal end 2004). The closure system 2006 can automatically transition to the closed configuration when a corresponding connector is detached from the connector 2002 (e.g., from the distal end 2004).

The closure system 2006 can include a valve member (e.g., which can be similar to the valve member 116 in the ′712 Publication). The valve member can move axially between a closed configuration or distal position, which can seal an inside of the male luer, and an open configuration or proximal position, which can open the male luer. FIG. 2 shows the valve member in the closed configuration or distal position. In some cases, as another connector (e.g., sometimes referred to as a mating connector) is coupled to the distal end 2004, the other (e.g., mating) connector can push the valve member proximally, such as by pressing on arms of the valve member that extend into a space between the male luer and the shroud. A biasing member (e.g., similar to the resilient member 118 of the ′712 Publication) can bias closure system 2006 to the closed configuration, such as by biasing the valve member to the distal or closed position.

The connector 2002 can have a housing 2010 (e.g., which can be similar to the male housing 123 of the ′712 Publication). The housing 2010 can be generally cylindrical and can define an interior space therein. The interior space can contain at least part of the distal end 2004, the closure system 2006, the fluid pathway 2008, and/or the proximal end 2014. The housing 2010 can be configured to be gripped by the user, such as to rotate the connector 2002 relative to another medical implement (e.g., a mating connector) that is being coupled to the connector 2002.

The connector 2002 can include a rotation mechanism 2012, which can be configured to enable the proximal end 2014 of the connector 2002 to rotate relative to the housing 2010 and/or relative to the distal end 2004. In some embodiments, the rotation mechanism 2012 can be configured to permit rotation of the proximal end 2014 in a first direction (e.g., counterclockwise) and to impede rotation of the proximal end 2014 in a second direction (e.g., clockwise). In some embodiments, the rotation mechanism 2012 can have a free-spin configuration that enables the proximal end 2014 to rotate in both directions relative to the housing 2010 and/or distal end 2004.

In some embodiments, the housing 2010 can include a distal portion 2016 and a proximal portion 2018. The distal portion 2016 can be joined to the proximal portion 2018 so that they move together, and can be considered one functional component, in some embodiments. For example, the distal portion 2016 can coupled to the proximal portion 2018 by sonic welding, adhesive, screw threads, pins, snap fit features, friction fit features, or the like. The proximal portion 2018 of the housing 2010 can engage the proximal end member 2014. In some embodiments, the rotation mechanism 2012 can include elements that engage between the proximal portion 2018 of the housing 2010 and the proximal end member 2014.

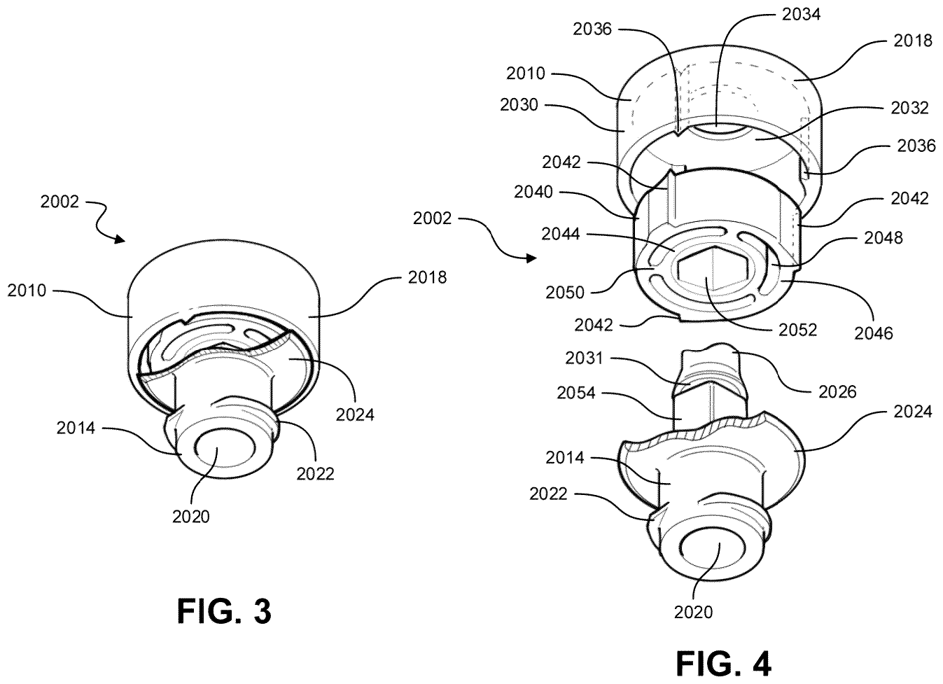

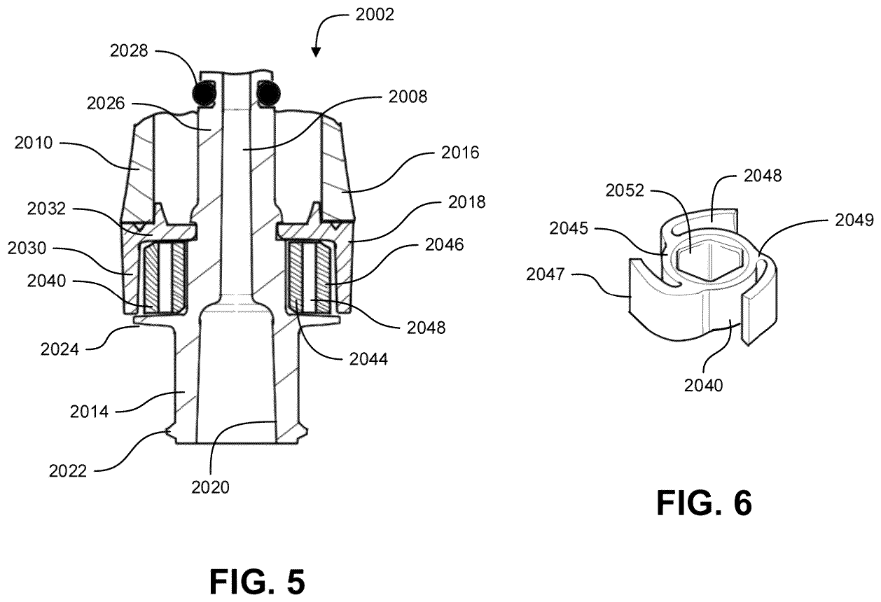

FIG. 3 is a partial view of an example embodiment of a connector 2002 with a one-way rotation mechanism. FIG. 4 is a partial exploded view of the connector 2002 of FIG. 3. FIG. 5 is a partial cross-sectional view of the connector 2002 of FIG. 3. The proximal end portion 2014 can include a female luer 2020, which can include a tapered bore that is configured to engage and seal with the outer taper of a male luer. External threads 2022 on the female luer 2020 can be configured to engage a luer lock feature (e.g., a shroud) associated with the male luer of another connector).

The proximal end portion 2014 can include a flange 2024. The female luer 2020 can extend proximally from the flange 2024. An interior lumen 2026 can extend distally from the flange 2024. A portion of the fluid pathway 2008 can extend from the female luer 2020 and through the interior lumen 2026. In some embodiments, a seal 2028 (e.g., an O-ring) can form a seal between the outside of the interior lumen 2026 and an inside surface of the valve member of the closure system 2006, which can be configured to slide axially relative to the interior lumen 2026.

The proximal portion 2018 of the housing 2010 can have a generally cylindrical sidewall 2030 and a flange 2032, which can extend inward from a distal end of the sidewall 2030. An opening 2034 can extend through the flange 2032, such as at a center of the flange 2032. The interior lumen 2026 can extend through the opening 2034. In some embodiments, a notch or groove 2031 on the interior lumen 2026 can engage the flange 2032, which can couple the proximal end portion 2014 to the housing 2010. In some embodiments, the engagement between the groove on the interior lumen 2026 and the flange 2032 on the housing 2010 can enable the proximal end portion 2014 to rotate relative to the housing 2010 but not move axially relative to the housing 2010. The inside of the sidewall 2030 can include one or more teeth 2036. In the illustrated embodiment, three teeth 2036 are distributed (e.g., evenly spaced) around the inside of the proximal portion 2018 of the housing 2010, although any suitable number of teeth 2036 (e.g., 1, 2, 3, 4, 5, 6, 8, 10, 12, or more) can be used.

The connector 2002 can include an engagement member 2040 (e.g., which can be an engagement disc or cylinder). The engagement member 2040 can be configured to fit in a space formed between the sidewall 2030 of the housing 2010 can the interior lumen 2026 of the proximal end portion 2014. The engagement member 2040 can include one or more teeth 2042, which can be configured to engage with the teeth 2036. In the illustrated embodiment, three teeth 2042 are distributed (e.g., evenly spaced) around the engagement member 2040, although any suitable number of teeth 2042 (e.g., 1, 2, 3, 4, 5, 6, 8, 10, 12, or more) can be used. The engagement member 2040 can have an inner ring 2044 and an outer ring 2046. The teeth 2042 can be formed on the outer surface of the outer ring 2046. Gaps 2048 can be disposed between the outer ring 2046 and the inner ring 2044 so that the teeth 2042 can be displaced inwardly. A gap 2048 can be disposed inward of each of the teeth 2042. Supports 2050 can couple the inner ring 2044 to the outer ring 2046, and the supports 2050 can separate the gaps 2048.

Each of the one or more teeth 2042 can have an engagement surface (e.g., which can extend generally radially outwardly). A line normal to the engagement surface can extend substantially tangentially from the engagement member 2040. Each of the one or more teeth 2040 can include a sliding surface (e.g., on a back side of the engagement surface). A line normal to the sliding surface can extend in a direction that is offset from a radially outward direction by an angle of about 5 degrees to about 45 degrees. Each of the one or more teeth 2036 on the housing 2010 can have an engagement surface (e.g., which can extend generally radially outwardly). A line normal to the engagement surface can extend substantially tangentially from the curvature of the housing 2010. Each of the one or more teeth 2036 can include a sliding surface (e.g., on a back side of the engagement surface). A line normal to the sliding surface can extend in a direction that is offset from a radially outward direction by an angle of about 5 degrees to about 45 degrees. The engagement surfaces of the teeth 2036 can face in an opposite direction as the engagement surfaces of the teeth 2042, so that the engagement surfaces abut against each other when the engagement member 2040 rotates relative to the housing 2010 in a second direction (e.g., clockwise). The sliding surfaces of the teeth 2036 and 2042 can slide over each other when the engagement member 2040 rotates relative to the housing 2010 in a first direction (e.g., counterclockwise). As the sliding surface press against each other, the gap 2048 can at least partially collapse so that the tooth 2042 moved radially inward to enable the sliding surface of the tooth 2042 to slide past the sliding surface of the tooth 2036. When the sliding surface pass each other, the tooth 2042 can snap radially outward as the gap 2048 returns to its default size. The teeth 2042 can be biased toward engagement with the teeth 2036 (e.g., radially outwardly).

The engagement member 2040 can have a keyed opening 2052, such as at a center of the engagement member 2040. A corresponding keyed portion 2054 on the interior lumen 2026 can be configured to engage the keyed opening 2052 on the engagement member 2040, so that the engagement member 2040 and the proximal end portion 2014 rotate together. In FIG. 4, a portion of the flange 2024 is cut away to show the keyed portion 2054 of the interior lumen 2026. The keyed opening 2052 and keyed portion 2054 can have six sides, although any suitable number of sides (e.g., 3, 4, 5, 6, 8, 10, 12, or more) can be used, or the keyed hole 2052 and keyed portion 2054 can have splines or any other engagement features so that they rotate together. In some embodiments, the keyed opening 2052 may be rigidly connected to the keyed portion 2054 through adhesive, friction fit features, snap fit features, or the like. The engagement member 2040 and the proximal end portion 2014 can be coupled by other suitable manners, such as by adhesive, friction fit features, snap fit features, or the like. The engagement member 2040 can operate as if it were functionally part of the proximal end portion 2014 in some embodiments. In some cases, the engagement member 2040 could be integrally formed with the proximal end portion 2014.

The connector 2002 can be configured to enable another connector (e.g., a mating connector) to couple to the proximal end portion 2014, and the connector 2002 can be configured to impede the other connector from being decoupled from the proximal end portion 2014. When another (e.g., mating) connector is being attached to the proximal end portion 2014 the other (e.g., mating) connector can be rotated in a tightening direction (e.g., clockwise) relative to the connector 2002 while the user grips the housing 2010. Friction between the other connector and the threading 2022 or the female luer 2020 can cause the proximal end portion 2014 to rotate in the tightening direction (e.g., clockwise) relative to the housing 2010. Once the engagement surfaces of the teeth 2036 and 2042 engage each other, the proximal end portion 2014 can be impeded from rotating in the tightening direction (e.g., clockwise) relative to the housing 2010. Stated another way, the teeth 2036 and 2042 can cause the housing 2010 and proximal end portion 2014 to rotate together in the tightening direction (e.g., clockwise). The user can twist the other connector and/or the housing 2010 of the connector 2002 so that the other connector engages the proximal end portion 2014, such as with a male luer tip that engages (e.g., sealingly engages) the female luer 2020 (e.g., to form a fluid pathway).

If rotation in the loosening direction (e.g., counter clockwise) is applied between the connector 2002 and the other connector, the sliding surface of the teeth 2036 and 2042 can slide over each other, and the teeth 2042 can be displaced inward, so that the proximal end portion 2014 (e.g., and the other connector attached thereto) can be permitted to rotate relative to the housing 2010. Thus, applying rotation in the loosening direction can merely cause the connectors to spin relative to each other, without loosening and/or without disengaging.

In some embodiments, the flange 2024 can be exposed and can be used to disengage the other connector from the connector 2002. For example, the housing 2010 can end without covering the flange 2024. The flange 2024 can be disposed proximally of the housing 2010. In some embodiments, the user can grip or otherwise engage the flange 2024 to hold the proximal end portion 2014 while the other connector is rotated in the loosening direction relative to the proximal end portion 2014. Thus, the connector 2002 can enable disengagement from the other connector by affirmative disengagement action by the user. The connector 2002 can impede unintended disengagement such as by bumping of the connectors or line twist, etc.

FIG. 6 shows an alternative example embodiment of an engagement member 2040. The engagement member 2040 can include a body portion 2045. A keyed opening 2052 can extend through the body portion 2045 and can engage a corresponding keyed portion 2054 on the proximal end portion 2014. The engagement member 2040 can include one or more pawls 2047, which can be configured to engage the teeth 2036 on the housing 2010. Each of the pawls 2047 can extend from a connection point 2049 on the body 2045. The pawl 2047 can be cantilevered to form a gap 2048 between the pawl 2047 and the body 2045. The pawl 2047 can be curved so that an end portion of the pawl tracks the same general curvature of the body 2045. The thickness of the pawls may be reduced in order to create a flexible attachment or pivot, such as a living hinge, at or near the connection point 2049 to the engagement cylinder 2040. Each of the pawls 2047 can have an engagement surface and a sliding surface, similar to the teeth 2042. A line normal to the engagement surface can extend generally tangentially to the curvature of the engagement member 2040. The sliding surface can be angled so that a line normal to the sliding surface can extend in a direction that is offset from a radially outward direction by an angle of about 5 degrees to about 45 degrees.

When the engagement member 2040 is rotated in a tightening direction (e.g., clockwise) relative to the housing 2010, such as when coupling another connector to the proximal end portion 2014, the engagement surfaces on the pawls 2047 can engage with the engagement surfaces of the teeth 2036 so that the engagement member 2040 does not rotate relative to the housing 2010 (e.g., to enable attachment of the other connector to the proximal end portion 2014). When the engagement member 2040 is rotated in a loosening direction (e.g., counter clockwise) relative to the housing, such as when the other connector is turned in a loosening direction, the sliding surfaces of the pawls 2047 can slide against the sliding surfaces of the teeth 2036, and the pawls 2047 can flex inwardly to enable the engagement member 2040 to rotate relative to the housing 2010.

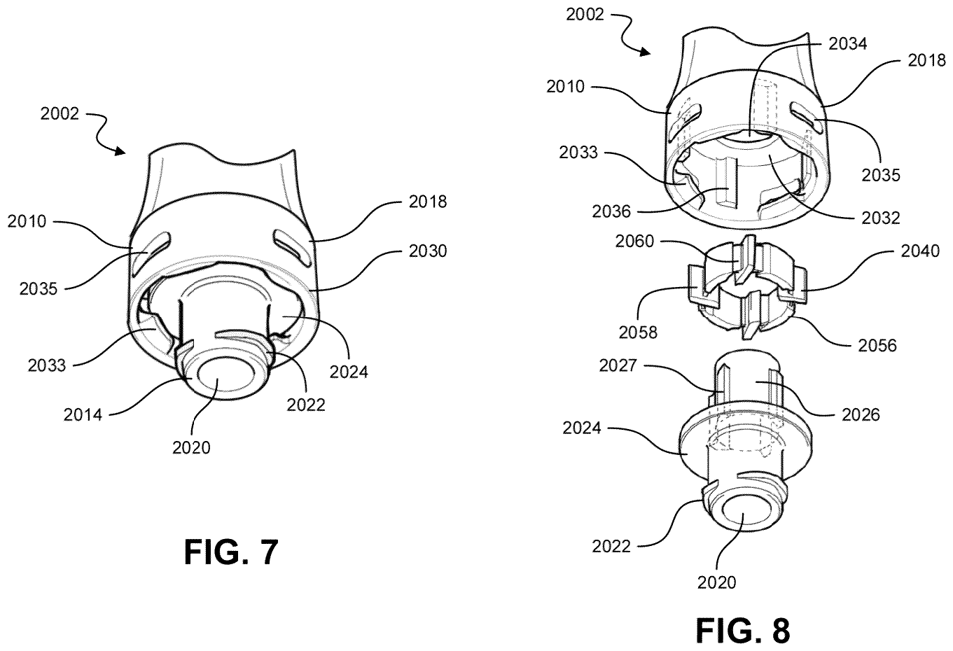

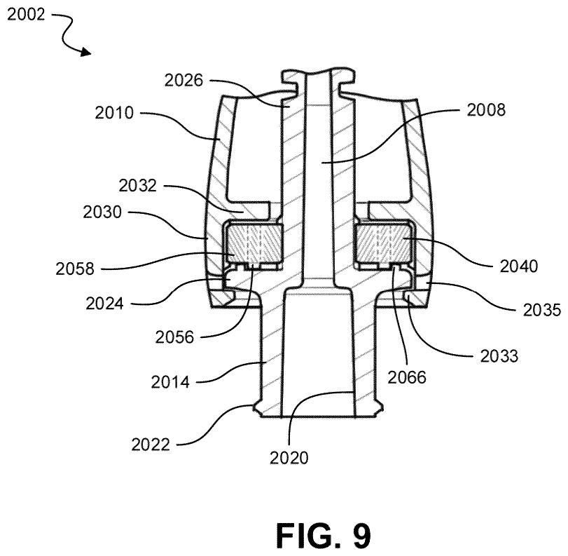

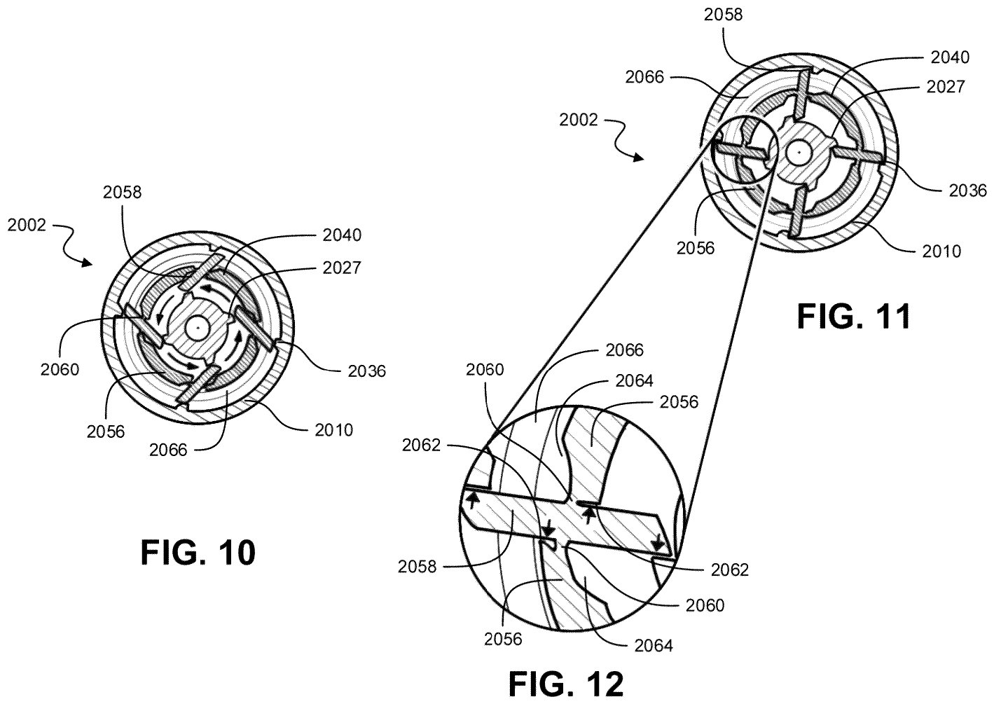

FIG. 7 is a partial perspective view of an example embodiment of a connector 2002. FIG. 8 is a partial exploded view of the example connector 2002 of FIG. 7. FIG. 9 is a partial cross-sectional view of the connector 2002 of FIG. 7. FIG. 10 is a cross-sectional view through the rotation mechanism of the connector 2002 of FIG. 7, showing rotation in a first direction. FIG. 11 is a cross-sectional view through the rotation mechanism of the connector 2002 of FIG. 7, showing rotation in a second direction. FIG. 12 is a detailed view of part of the cross-sectional view of FIG. 11. In some aspects, the connector 2002 of FIGS. 7-12 can be similar to the connectors of FIGS. 3-6, except as described.

The housing 2010 can have a generally cylindrical sidewall 2030, with a flange 2032 (e.g., an internal flange) that extends inwardly from the side wall 2030, with an opening 2034 in the flange 2032. An interior lumen 2026 of the proximal end portion 2014 can pass through the opening 2034 in the internal flange 2032. One or more tabs 2033 can extend inward from the sidewall 2030, such as at or near the proximal end of the housing 2010. The flange 2024 of the proximal end portion 2014 can be recessed into the housing 2010. The flange 2024 can be positioned on a distal side of the one or more tabs 2033, so that the tabs 2033 can hold the flange 2024 inside the housing 2010 (e.g., without impeding rotation of the proximal end portion 2014 relative to the housing 2010). The tabs 2033 and/or the flange 2024 can be configured to flex sufficiently to enable the flange 2024 to be inserted distally past the one or more tabs 2033, such as for a snap fit engagement. The housing can include openings 2035 formed in the sidewall 2030, which openings 2035 can at least partially align with the tabs 2033. The openings 2035 can facilitate flexing of the housing 2010 (e.g., the sidewall 2030) so that the tabs 2033 can be displaced sufficiently for the flange 2024 of the proximal end portion 2014 to be inserted into the housing 2010. The proximal-facing sides of the tabs 2033 can be angled, and/or the distal-facing side of the flange 2024 can be angled, so that when the flange 2024 is pressed distally against the tabs 2033, the tabs can be displaced outwardly.

The inside surface of the sidewall 2030 can include one or more protrusions or teeth 2036. The outside surface of the interior lumen 2026 of the proximal end portion 2014 can include one or more protrusions or teeth 2027. In the illustrated embodiment, 4 protrusions or teeth are shown, but any suitable number (e.g., 1, 2, 3, 4, 6, 8, 10, 12, or more) can be used. The protrusions 2036 on the housing 2010 are not configured to engage the protrusions 2027 on the proximal end portion 2014 directly. Rather, an engagement member 2040 can be positioned between the protrusions 2036 on the housing 2010 and the protrusions 2027 on the proximal end portion 2014.

The engagement member 2040 can have a plurality of body portions 2056, which can each be an arcuate segment, which can be positioned along an arcuate path. A plurality of arms 2058 can be disposed between the body portions 2056. The arms 2058 can be rotatably coupled to the body portions 2056 at junctions 2060 (e.g., which can be living hinges). For example, the junctions 2060 can have a thickness that is less than the thickness of the body portion(s) 2056 and/or that is less than the thickness of the arm(s) 2058. The junctions 2060 can be configured to permit the arms 2058 to pivot or rotate between positions. The arm(s) 2058 can have an outer portion, which can be disposed outward of the junction 2060, and which can be configured to selectively engage the protrusion(s) 2036 on the housing 2010. The arm(s) 2058 can have an inner portion, which can be disposed inward of the junction 2060, and which can be configured to selectively engage the protrusion(s) 2027 on the proximal end portion 2014.

The arms 2058 can have a rotation configuration (e.g., shown in FIG. 10), which can enable the proximal end portion 2014 rotate in a first direction relative to the housing 2010, and a locking configuration (e.g., shown in FIGS. 11 and 12), which can impede the proximal end portion 2014 from rotating in a second direction relative to the housing 2010. The arms 2058 can extend generally radially in the locking configuration. The arms 2058 in the locking configuration can extend along a direction that is offset from a radial direction by about 30 degrees, about 20 degrees, about 15 degrees, about 10 degrees, about 5 degrees, about 3 degrees, about 1 degree, or about 0 degrees, or any ranges or values therebetween, although other configurations could be used. The arms 2058 in the rotation configuration can extend along a direction that is offset from the radial direction by about 15 degrees, about 20 degrees, about 25 degrees, about 30 degrees, about 35 degrees, about 45 degrees, about 50 degrees, about 55 degrees, about 60 degrees, about 65 degrees, about 70 degrees, about 75 degrees, or any values or ranges therebetween, although other angles could be used in some cases. The arms 2058 can rotate between the rotation configuration and the locking configuration by an angle of at least about 15 degrees, about 20 degrees, about 25 degrees, about 30 degrees, about 35 degrees, about 40 degrees, about 45 degrees, about 50 degrees, about 55 degrees, or about 60 degrees, or any values or ranges therebetween.

A first junction 2060 (e.g., a first living hinge) can be on a first side of the arm 2058 and can join the arm 2058 to a first body portion 2056. A second junction 2060 (e.g., a second living hinge) can be on a second side of the arm 2058 and can join the arm 2058 to a second body portion 2056. The first body portion 2056 on the first side of the arm 2058 can have a stopper surface 2062 on a first (e.g., radially inward) side of the junction 2060. The second body portion 2056 on the second side of the arm 2058 can have a stopper surface 2062 on a second (e.g., radially outward) side of the junction 2060. The first body portion 2056 on the first side of the arm 2058 can have a recess 2064 on a second (e.g., radially outward) side of the junction 2060. The second body portion 2056 on the second side of the arm 2058 can have a recess 2064 on a first (e.g., radially inward) side of the junction 2060.

As shown in FIGS. 11 and 12, when the proximal end portion 2014 rotates in a tightening direction (e.g., clockwise) relative to the housing 2010, the protrusions 2027 can press on the second sides of the arms 2058, which can cause arms 2058 to rotate in a first direction (e.g., counter clockwise) toward the locking configuration. The first side of the arm 2058 can abut against the stopper surface 2062 on the first body portion 2056, and/or the second side of the arm 2058 can abut against the stopper surface 2062 on the second body portion 2056, to impede the arm 2058 from rotating past the locking position shown in FIGS. 11 and 12. The arm 2058 can extend generally radially in the locking configuration. The radially inward end of the arm 2058 can abut against the protrusion 2027 on the proximal end portion 2014, and the radially outward end of the arm 2058 can abut against the protrusion 2036 on the housing 2010. The arm 2058 can engage the protrusions 2027 and 2036 to impede the proximal end portion 2014 from rotating in the tightening direction (e.g., clockwise) relative to the housing 2010. The protrusions 2027 and/or 2036 can be teeth with engagement surfaces and sliding surfaces similar to those described in connection with FIG. 4, while in some embodiments, the protrusions 2027 and/or 2036 can omit the angled sliding surfaces. For example, the arms 2058 of the engagement member 2040 can fold or pivot to provide sliding surfaces.

As shown in FIG. 10, when then proximal end portion 2014 rotates in a loosening direction (e.g., counterclockwise), the protrusions 2027 can press on the first side of the arms 2058, which can cause the arms 2058 to pivot towards the rotating configuration. The arms 2058 can fold or pivot into the recesses 2064 formed in the body portions 2056, so that the arms 2058 can disengage from the protrusions 2027 and/or 2036 so that the proximal end portion 2014 can rotate relative to the housing 2010 in the loosening direction (e.g., counterclockwise).

From the configuration shown in FIGS. 11 and 12, if the user tries to rotate the proximal end portion 2014 in the tightening direction (e.g., clockwise), the arms 2058 will press against the stopper surfaces 2062, and the protrusions 2027 and 2036 will press against the arms 2058, to impede the proximal end portion 2014 from rotating. If the user rotates the proximal end portion 2014 in the loosening direction (e.g., counter clockwise), the protrusions 2027 and/or 2036 will fold the arms 2058 (e.g., into the recesses 2064), so that the protrusions 2027 and/or 2036 can move past the arms 2058 to enable rotation of the proximal end portion 2014 relative to the housing 2010. Similar to other embodiments disclosed herein, the proximal end portion 2014 can be locked against rotation relative to the housing 2010 during coupling of another connector to the proximal end portion 2014, and the proximal end portion 2014 can spin relative to the housing 2010 to impede decoupling of the other connector from the proximal end portion 2014.

In some embodiments, a ridge 2066 can be disposed on a distal side of the flange 2024, such as along an arcuate path. The body portions 2056 of the engagement member 2040 can fit inside of the ridge 2066. The ridge 2066 can be configured to hold the engagement member 2040 in a substantially concentric position relative to the housing 2010, proximal end portion 2014, and/or other components of the connector 2002. The body portions 2056 can extend proximally further than the arms 2058, so that the arms 2058 can pass over the ridge 2066.

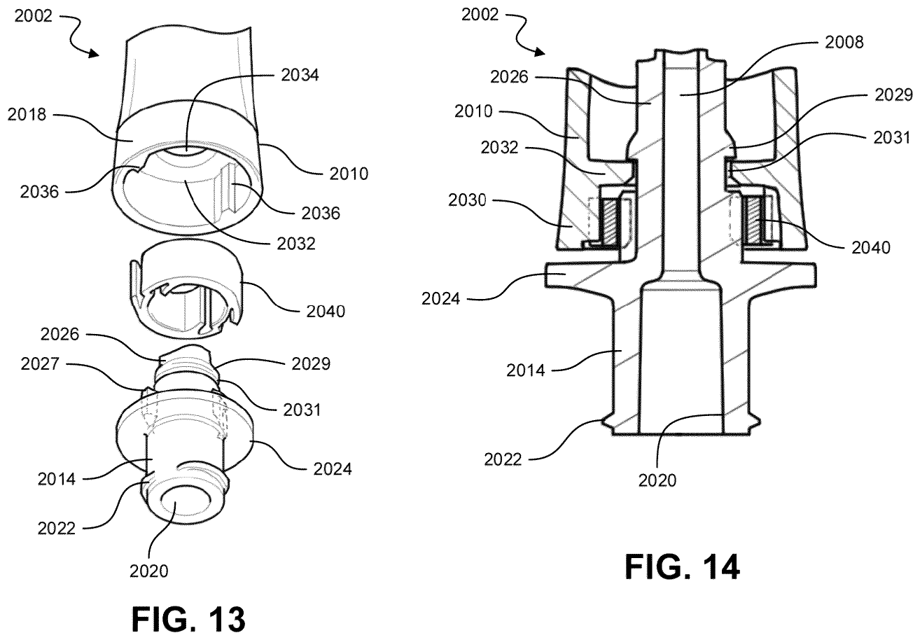

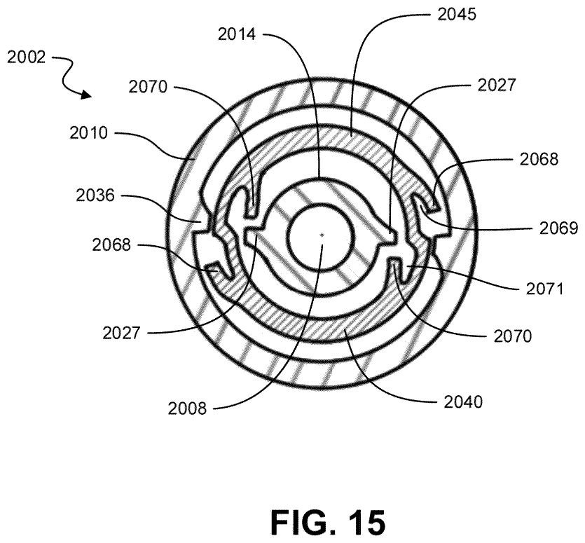

FIG. 13 is a partial exploded view of another example embodiment of a connector 2002. FIG. 14 is a partial cross-sectional view of the example connector 2002 of FIG. 13. FIG. 15 is a cross-sectional view taken through the rotation mechanism of the example connector 2002 of FIG. 13. The connector 2002 can have a proximal end portion 2014 with a female luer 2020, a flange 2024, and an interior lumen 2026. The proximal end portion 2014 can include protrusions 2027, such as extending outward from the interior lumen 2026 adjacent to the flange 2024. The protrusions 2027 can be teeth with engagement surfaces on one side and sliding surfaces on the other side. Two protrusions 2027 are shown, but any suitable number (e.g., 1, 2, 3, 4, 6, 8, 10, 12, or more) can be used.

The housing 2010 can include a sidewall 2030, a flange 2032 that extends inwardly from the side wall 2030, and an opening 2034 through the flange 2032. A distal portion of the interior lumen 2026 can extend through the opening 2034 in the flange 2032. A ridge 2029 or one or more protrusions can extend outwardly from the interior lumen 2026 and can be configured to fit through the opening 2034 (e.g., as a snap fit engagement) and to engage with the flange 2032 to couple the proximal end portion 2014 to the housing 2010. The flange 2032 can fit into a groove 2031 or recess, which can be formed between the ridge 2029 and the protrusion(s) 2027. The distal side of the ridge 2029 or protrusion(s) can be angled to facilitate insertion of the ridge 2029 or protrusion(s) through the opening 2034. The proximal side of the ridge 2029 or protrusion(s) can extend laterally, to provide an engagement surface that engages with a distal side of the flange 2032. The housing 2010 can include protrusions 2036, such as extending inwardly from the sidewall 2030. The protrusions 2036 can be teeth with engagement surfaces on one side and sliding surfaces on the other side. Two protrusions 2036 are shown, but any suitable number (e.g., 1, 2, 3, 4, 6, 8, 10, 12, or more) can be used.

The connector 2002 can include an engagement member 2040, which can have a body 2045 having a generally circular shape. The engagement member 2040 can have outward pawls 2068, which can be configured to engage the protrusions 2036 on the housing 2010, and inward pawls 2070, which can be configured to engage the protrusions 202 on the proximal end portion 2014. When the proximal end portion 2014 is rotated in the tightening direction (e.g., clockwise in FIG. 15), the protrusions 2027 can abut against the ends of the inner pawls 2070, so that the engagement member 2040 rotates with the proximal end portion 2014 until the outer pawls 2068 abut against the protrusions 2036 on the housing 2010. If the user tries to rotate the proximal end portion 2014 further in the tightening direction, the protrusions 2027 will press against the inner pawls 2070 and the outer pawls 2068 will press against the protrusions 2036 on the housing 2010 to impede rotation of the proximal end portion 2014 relative to the housing 2010. When another connector is being coupled to the proximal end portion 2014, the engagement member 2040 can engage the protrusions 2027 and 2036 so that the proximal end portion 2014 does not rotate relative to the housing 2010.

The engagement member 2040 can have a gap 2069 between the outer pawl 2068 and the body 2045, which can enable the outer pawl 2068 to flex inward towards the body 2045. The engagement member 2040 can have a gap 2071 between the inner pawl 2070 and the body 2045, which can enable the inner pawl 2070 to flex outward towards the body 2045. When the proximal end portion 2014 is rotated in a loosening direction (e.g., counter clockwise in FIG. 15), the protrusions 2027 can press on an inward facing side of the inner pawls 2070 to deflect the inner pawls 2070 outward (e.g., by at least partially collapsing the gaps 2071), which can permit the protrusions 2027 to rotate past the inner pawls 2070, so that the proximal end portion 2014 can spin freely relative to the housing 2010. In some cases, the proximal end portion 2014 can drive the engagement member 2040 in the loosening direction (e.g., counter clockwise in FIG. 15), so that the outside surfaces of the outer pawls 2068 can slide along the protrusions 2036 on the housing 2010, which can deflect the outer pawls 2068 inwardly (e.g., by at least partially collapsing the gaps 2069), such as to permit the protrusions 2036 to pass over the outer pawls 2068. The connector 2002 can be configured so that the proximal end portion 2014 spins freely relative to the housing 2010 and engagement member 2040, or the connector 2002 can be configured so that the proximal end portion 2014 and engagement member 2040 spin freely relative to the housing 2010. In some embodiments, the outer pawls 2068 can be configured to deflect more easily than the inner pawls 2070, such as to let the protrusions 2036 on the housing rotate past the outer pawls 2068. In some embodiments, the inner pawls 2070 can be configured to deflect more easily than the outer pawls 2068, such as to let the protrusions 2027 rotate past the inner pawls 2070.

In some embodiments, the proximal end portion 2014 can be configured to spin freely relative to the housing 2010 in the tightening direction (e.g., clockwise in FIG. 15) for a range of motion before the rotation mechanism engages to impede the proximal end portion 2014 from spinning further relative to the housing 2010. The connector 2002 can be configured to have a maximum free spin range in the tightening direction of about 30 degrees, about 45 degrees, about 60 degrees, about 75 degrees, about 90 degrees, about 105 degrees, about 120 degrees, about 135 degrees, about 150 degrees, about 165 degrees, about 180 degrees, about 195 degrees, about 210 degrees, about 225 degrees, about 240 degrees, about 255 degrees, about 270 degrees, about 285 degrees, about 300 degrees, about 315 degrees, about 330 degrees, about 345 degrees, or about 360 degrees, or any values or ranges therebetween. With reference to FIG. 15, the proximal end portion 2014 can rotate a maximum of about 180 degrees before the protrusions 2027 engage the inner pawls 2070 on the engagement member 2040, and the proximal end portion 2014 and engagement member 2040 can rotate together by a maximum of about 180 degrees before the outer pawls 2068 of the engagement member 2040 engage with the protrusions 2036 on the housing 2010. Any suitable number of teeth, protrusions, or pawls can be used to provide the free spin ranges disclosed herein.

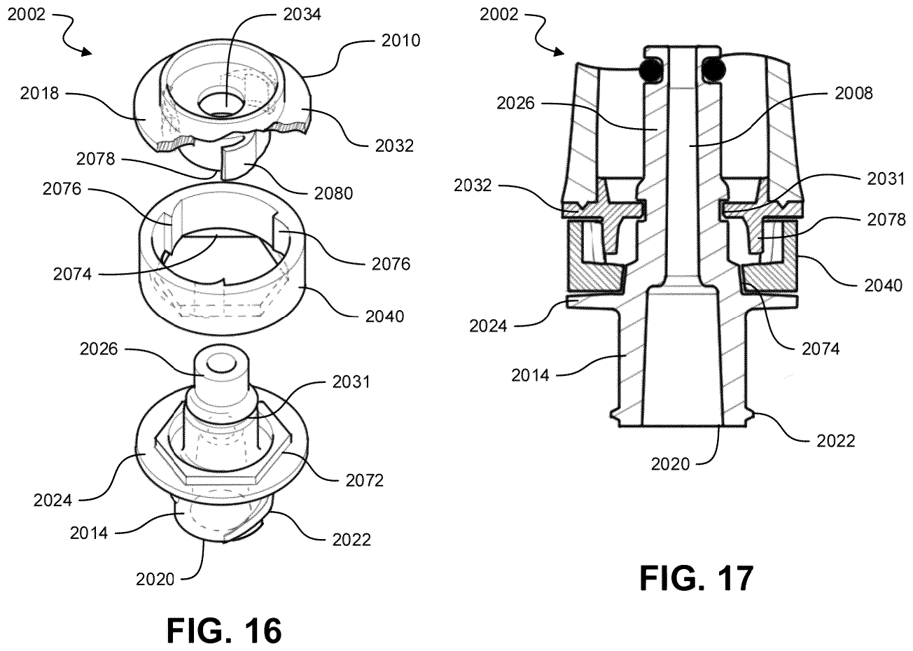

In the embodiments of FIGS. 3-15, the engagement mechanism (e.g., teeth or pawls) on the housing 2010 can be positioned radially outward of the engagement mechanism (e.g., teeth or pawls) on the proximal end portion 2014. In some embodiments, the engagement mechanism (e.g., teeth or pawls) on the housing 2010 can be positioned radially inward of the engagement mechanism (e.g., teeth or pawls) on the proximal end portion 2014, as shown in FIGS. 16 and 17, for example. FIG. 16 shows a partial exploded view of an example embodiment of a connector 2002. FIG. 17 shows a partial cross-sectional view of the connector 2002 of FIG. 16.

The connector 2002 can have a proximal end portion 2014 that includes a female luer 2020, external threading 2022, a flange 2024, and an interior lumen 2026. The distal side of the flange 2024 can have a keyed portion 2072. The engagement member 2040 can have a body 2045, which can have a generally circular shape. The engagement member 2040 can have a keyed portion 2074 on a proximal end thereof, which can be configured to engage the keyed portion 2072 on the proximal end portion 2014. For example, the keyed portion 2072 can be a raised portion (e.g., in the shape of a hexagon), and the keyed portion 2074 can be a recess (e.g., in the shape of a hexagon), which can be configured to receive the raised structure of the keyed portion 2072 therein, so that the engagement member 2040 rotates with the proximal end portion 2014. Many other configurations could be used. Although a hexagonal keyed shape is used, various other polygonal shapes could be used, or splines, or any other keyed structures. In some cases, the flange 2024 could include a keyed recess that is configured to receive a keyed protrusion on the engagement member 2040.

The body portion 2045 can form an external sidewall of the connector 2002. The body portion 2045 can have a generally cylindrical shape, with an open center. The engagement member 2040 can have internal teeth 2076 or protrusions, which can extend inwardly from the body portion 2045. The teeth 2076 can be disposed distally of the keyed portion 2074. The housing 2010 can include a proximal housing portion 2018 that has a flange 2032 and an opening 2034 through the flange 2032. The flange 2032 can be configured to engage the groove 2031 on the interior lumen 2026, such as to couple the proximal end portion 2014 to the housing 2010. The interior lumen 2026 can extend through the open center of the engagement member 2040 and through the opening 2034 in the flange 2032 of the housing 2010.

The housing 2010 can have one or more pawls 2080, which can be configured to engage the teeth 2076 on the engagement member 2040. A proximal body portion 2078 can extend proximally from the flange 2032. The proximal body portion 2078 can be generally cylindrical in shape. The opening 2034 can extend through the proximal body portion 2078. The pawls 2080 can extend outwardly from the proximal body portion 2078. The pawls 2080 can be curved, such as so that the ends of the pawls 2080 generally follow the curvature of the proximal body portion 2078. A line normal to the surfaces at the ends of the pawls 2080 can extend generally tangentially. The pawls 2080 can have engagement surfaces that extend generally radially, such as to engage the engagement surfaces on the teeth 2076. FIG. 16 shows three pawls 2080 and three teeth 2076, but any suitable number (e.g., 1, 2, 3, 4, 6, 8, 10, 12, or more) could be used.

When the proximal end portion 2014 is rotated in a tightening direction (e.g., clockwise in FIG. 16), such as when attaching another connector to the proximal end portion 2014, the engagement member 2040 can rotate with the proximal end portion 2014 due to the engagement between the keyed portions 2072 and 2074. The proximal end portion 2014 and engagement member 2040 can rotate until the teeth 2076 engage with the pawls 2080 on the housing 2010 to impede further rotation of the proximal end portion 2014 and the engagement member 2040 relative to the housing 2010. The teeth 2076 can have engagement surfaces and sliding surfaces, similar to other embodiments disclosed herein. The pawls 2080 can have engagement ends configured to abut against the engagement surfaces of the teeth 2076 to impede relative rotation. When engaged, the housing 2010 can rotate with the proximal end portion 2014. Thus, the user can hold the housing 2010, which can impede the proximal end portion 2014 from rotating while another connector is rotated onto the proximal end portion 2014 (e.g., in the tightening direction).

When the proximal end portion 2014 is rotated in the loosening direction (e.g., clockwise in FIG. 16), the engagement member 2040 can rotate along with the proximal end portion 2014 due to the engagement between the keyed portions 2072 and 2074. The sliding surfaces on the teeth 2076 of the engagement member 2040 can slide against the outside or back surfaces of the pawls 2080 of the housing 2010, which can deflect the pawls 2080 inwardly so that the teeth 2076 can rotate past the pawls 2080. Thus, the proximal end portion 2014 can be configured to spin freely relative to the housing 2010 in the loosening direction. Thus, if a user rotates another connector that is attached to the proximal end portion 2014 in the loosening direction, the proximal end portion 2014 can rotate along with the other connector, which can impede the other connector from detaching form the proximal end portion 2014. In some embodiments, the user can grip or otherwise manipulate the flange 2024 to hold the proximal end portion 2014 against rotation so that the other connector can be detached therefrom.

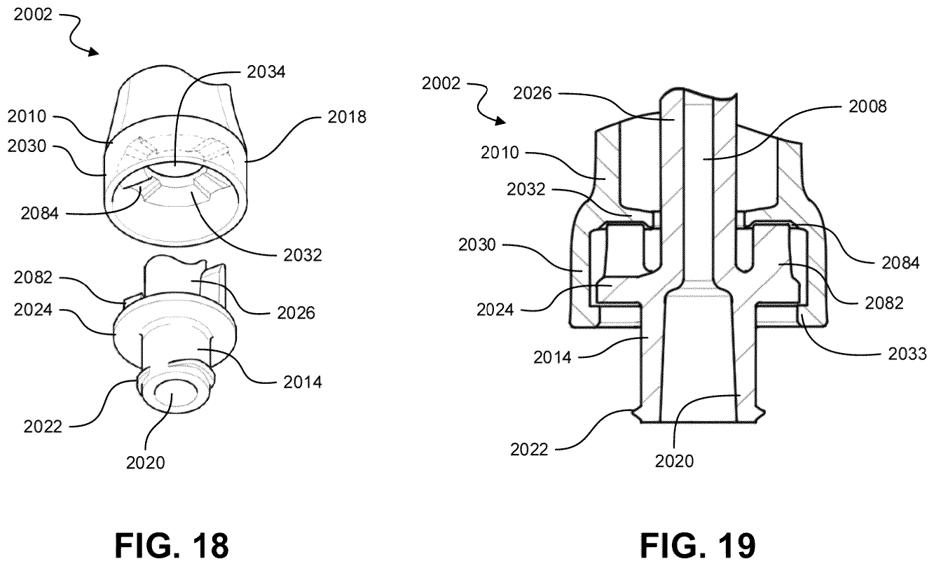

FIG. 18 is a partial exploded view of another example embodiment of a connector 2002. FIG. 19 is a partial cross-sectional view of the example connector 2002 of FIG. 18. The connector 2002 can have a proximal end portion 2014 that includes a female luer 2020, external threading 2022, a flange 2024, and an interior lumen 2026. The proximal end portion 2014 can have pawls 2082 that extend distally from the flange 2024.

The connector 2002 can have a housing 2010 that includes a sidewall 2030, which can be generally cylindrical in shape. A flange 2032 can extend inwardly from the sidewall 2030 of the housing 2010. An opening 2034 can extend through the flange 2032. The interior lumen 2008 can extend through the opening 2034. The housing can have one or more tabs 2033 for securing the proximal end portion 2014 to the housing 2010. The one or more tabs 2033 can extend inward from the sidewall 2030, such as at or near the proximal end of the housing 2010. The flange 2024 of the proximal end portion 2014 can be recessed into the housing 2010. The flange 2024 can be positioned on a distal side of the one or more tabs 2033, so that the tabs 2033 can hold the flange 2024 inside the housing 2010. The tabs 2033 and/or the flange 2024 can be configured to flex sufficiently to enable the flange 2024 to be inserted distally past the one or more tabs 2033, such as for a snap fit engagement.

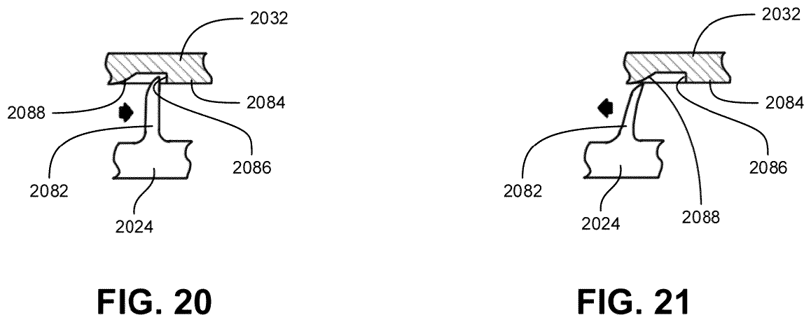

The proximal side of the flange 2032 on the housing can have engagement features 2084 that are configured to engage the pawls 2082. The engagement features 2084 can include teeth, protrusions, or recesses. The engagement features 2084 can have engagement surfaces 2086 and sliding surfaces 2088. A line normal to the engagement surfaces 2086 can extend laterally, or orthogonal to a longitudinal axis of the connector 2002. The engagement surfaces 2086 can be configured to abut against engagement sides of the pawls 2082, such as to impede rotation of the proximal end portion 2014 relative to the housing 2010. The engagement side of the pawl 2082 can have a surface that is substantially parallel to the engagement surface 2086. With reference to FIG. 20, the pawl 2082 can have an unflexed or undeformed position when it is located between engagement features 2084. When the proximal end portion 2014 is rotated in a tightening direction (e.g., clockwise in FIG. 18 or to the right in FIG. 20), the engagement side of the pawl 2082 can abut against the engagement surface 2086 to impede the proximal end portion 2014 from rotating further in the tightening direction.

The engagement features 2084 can have sliding surfaces 2088, such as opposite the engagement surfaces 2086. The sliding surfaces 2088 can be angled relative to the engagement surfaces 2086 by about 15 degrees to about 75 degrees, or by about 30 degrees to about 60 degrees. The pawls 2082 can have sliding surfaces on the sides of the pawls 2082 that face the sliding surfaces 2088 of the engagement features 2084 on the housing 2010. The pawls 2082 can have a tapered thickness at the proximal ends. The sliding surfaces on the pawls 2082 can be angled relative to the engagement surfaces 2086 by about 15 degrees to about 75 degrees, or by about 30 degrees to about 60 degrees. The sliding surfaces on the pawls 2082 and the engagement features 2084 can facilitate or permit rotation of the proximal end portion 2014 relative to the housing 2010 in the loosening direction (e.g., counterclockwise in FIG. 18 or to the left in FIG. 21). When the proximal end portion 2014 moves in the loosening direction relative to the housing 2010, the sliding surfaces of the pawls 2082 can slide against the sliding surfaces 2088 of the engagement features 2084, which can cause the pawls 2082 to flex, so that the pawls 2082 can pass over the engagement features 2084. Thus, the connector 2002 can be configured to permit the proximal end portion 2014 to rotate in the loosening direction.

In some cases, if an amount of torque above a threshold is applied to the proximal end portion 2014 in the tightening direction, the pawls 2082 can flex enough for the pawls 2082 to slide past the engagement surface 2086. Thus, by applying torque above the threshold, the proximal end portion 2014 can be rotatable in the tightening direction. The threshold of torque or force that rotates the proximal end portion 2014 in the tightening direction can be higher than the threshold amount of torque or force that rotates the proximal end portion 2014 in the loosening direction relative to the housing 2010. The threshold of torque or force for rotating the proximal end portion 2014 in the tightening direction can be higher than the amount of torque or force that would be applied to fully couple another connector to the proximal end portion. The threshold of torque or force for rotating the proximal end portion 2014 in the loosening direction can be lower than the amount of torque or force that would be applied to decouple the other connector from the proximal end portion 2014.

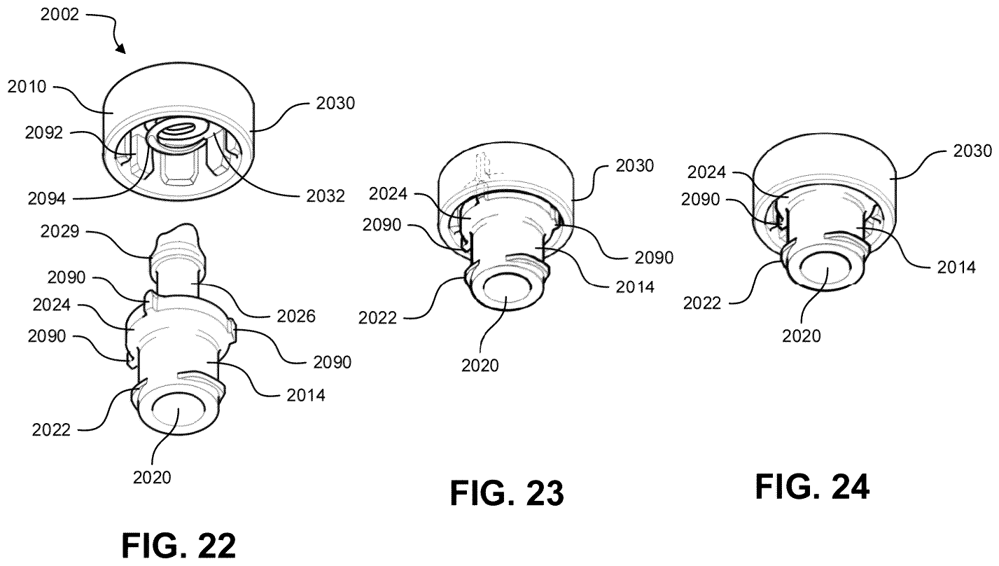

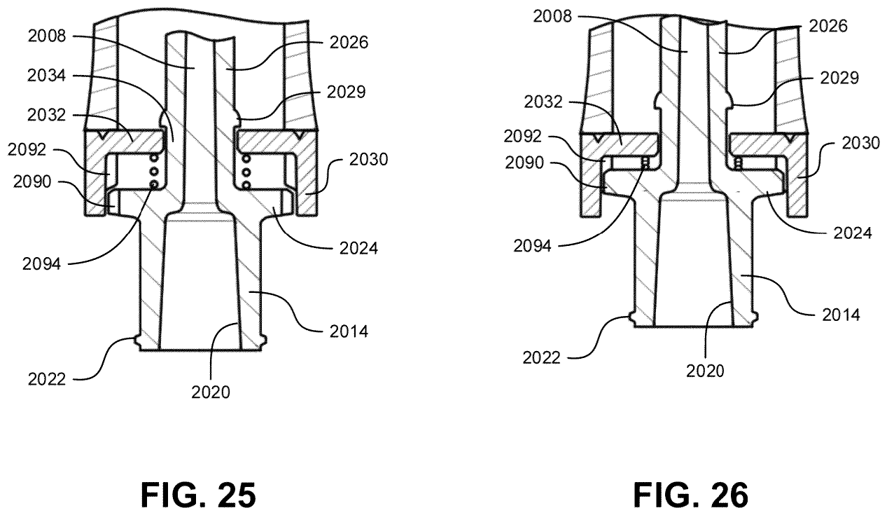

In some embodiments, the connector 2002 can have a free-spin configuration that permits the proximal end portion 2014 to spin freely in both directions relative to the housing 2010. The connector 2002 can have an engaged configuration that impedes rotation of the proximal end portion 2014 relative to the housing 2010 in at least one direction, or in both directions. FIG. 22 shows a partial exploded view of another example embodiment of a connector 2002. FIG. 23 shows a partial perspective view of the example connector 2002 of FIG. 22 in a free-spin configuration. FIG. 24 shows a partial perspective view of the example connector 2002 of FIG. 22 in an engaged configuration. FIG. 25 shows a partial cross-sectional view of the example connector 2002 of FIG. 22 in a free-spin configuration. FIG. 26 shows a partial cross-sectional view of the example connector 2002 of FIG. 22 in an engaged configuration.

The connector 2002 can have a proximal end portion 2014 that includes a female luer 2020, external threading 2022, a flange 2024, and an interior lumen 2026. The housing 2010 can have a sidewall 2030, a flange 2032 that extends inwardly from the sidewall 2030, and an opening 2034 through the flange 2032. The interior lumen 2026 can extend through the opening 2034 in the housing 2010. A ridge 2029 or one or more protrusions can extend outwardly from the interior lumen 2026 and can be configured to fit through the opening 2034 (e.g., as a snap fit engagement) and to engage with the flange 2032 to couple the proximal end portion 2014 to the housing 2010. The distal side of the ridge 2029 or protrusion(s) can be angled to facilitate insertion of the ridge 2029 or protrusion(s) through the opening 2034. The proximal side of the ridge 2029 or protrusion(s) can extend laterally, to provide an engagement surface that engages with a distal side of the flange 2032.

The proximal end portion 2014 can have one or more protrusions 2090, which can extend radially outward, such as from the flange 2024. The housing 2010 can have one or more corresponding recesses 2092, which can be shaped to receive the one or more protrusions 2090 therein. The housing 2010 can have protrusions with the recesses 2092 formed between the protrusions. The distal ends of the protrusions 2090 can be tapered and/or the proximal ends of the recesses 2092 can be flared, such as to facilitate sliding of the protrusions 2090 into the recesses 2092. A biasing member 2094, such as a coil spring, can be disposed between the flange 2032 on the housing 2010 and the flange 2024 on the proximal end portion 2014. The biasing member 2094 can bias the proximal end portion 2014 proximally relative to the housing 2010. The ridge 2029 can limit the range of motion of the proximal end portion 2014 in the proximal direction, such as by the ridge 2029 abutting against the flange 2032 on the housing 2010. The protrusions 2090 can be disposed outside the recesses 2092 when the proximal end portion 2014 is at the proximal position (e.g., shown in FIGS. 23 and 25). With the protrusions 2090 disengaged from the recesses 2092, the connector 2002 can be in a free-spin configuration, and the proximal end portion 2014 can be permitted to rotate freely in both directions.

The proximal end portion 2014 can be movable axially relative to the housing 2010. Pressing the proximal end portion 2014 axially in the distal direction can compress the biasing member 2094 and can cause the flange 2024 to move distally towards the flange 2032. As the proximal end portion 2014 moves distally, the protrusions 2090 can insert into the corresponding recesses 2092. With the protrusions 2090 engaged with the recesses 2092, rotation between the proximal end portion 2014 and the housing 2010 can be impeded. Rotating the proximal end portion 2014 in either direction would cause the protrusions 2090 to press against corresponding sides of the recesses 2092 so that the housing 2010. The user can move the proximal end portion 2014 to the engaged configuration (e.g., of FIGS. 24 and 26) when coupling another connector to the proximal end portion 2014. In the engaged configuration, the user can hold the housing and rotate the other connector relative to the housing 2010 and the proximal end portion 2014 so that the other connector can thread onto the proximal end portion 2014. Once the other connector is coupled to the proximal end portion 2014, the user can release the axial force on the proximal end portion 2014, so that the proximal end portion moves proximally to the free-spin configuration (e.g., of FIGS. 23 and 25). If the user then rotates the other connector in either direction, the proximal end portion 2014 will rotate with the other connector relative to the housing 2010. Thus, the connector 2002 can be configured to impede unintentional disconnecting of the other connector from the proximal end portion 2014. In some embodiments, the user could press the proximal end portion 2014 distally to engage the protrusions 2090 with the corresponding recesses 2092, and then it would be possible for the user to rotate the other connector in the loosening direction to remove the other connector from the proximal end portion 2014.

In some embodiments, the proximal end portion 2014 can move axially relative to the housing 2010, such as to transition between an engaged configuration and a disengaged configuration (e.g., as shown in FIGS. 22 to 26). In some embodiments, the connector 2002 can be configured to transition between an engaged configuration and a disengaged configuration without axial movement of the proximal end portion 2014 relative to the housing 2010. In some embodiments, the connector 2002 is configured to permit rotation of the proximal end portion 2014 relative to the housing 2010 in one or both directions without axial movement of the proximal end portion 2014 relative to the housing 2010. In some cases, the proximal end portion 2014 can be locked against axial movement relative to the housing 2010.

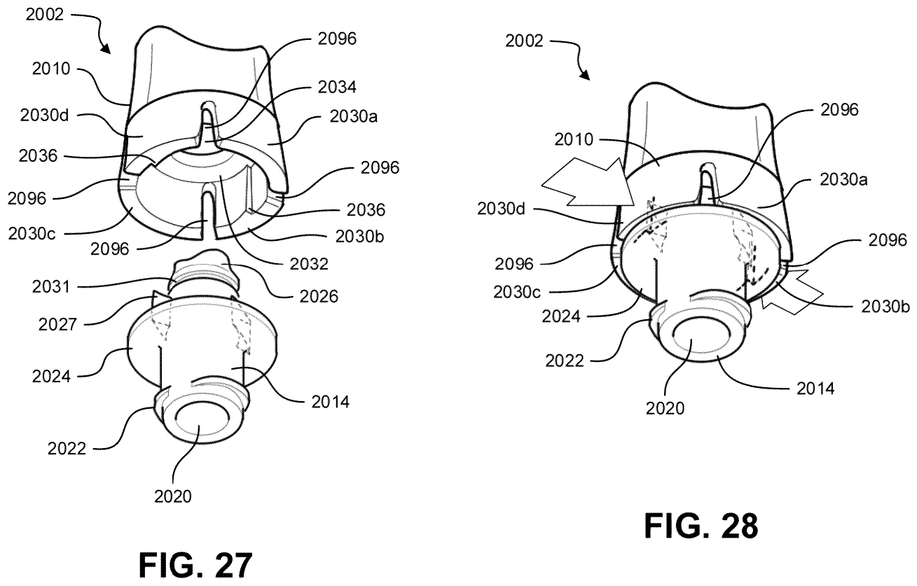

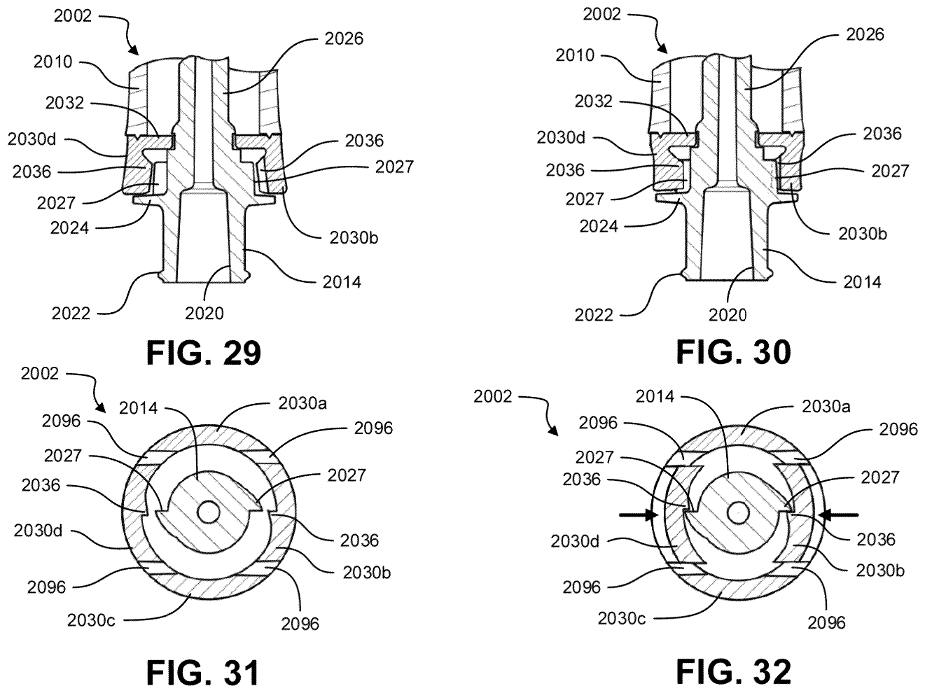

FIG. 27 shows a partial exploded view of another example embodiment of a connector 2002. FIG. 28 shows a partial perspective view of the example connector 2002 of FIG. 27. FIG. 29 shows a partial cross-sectional view of the example connector in a free-spin or disengaged configuration. FIG. 30 shows a partial cross-sectional view of the example connector in an engaged configuration. FIG. 31 shows a cross-sectional view through the rotation mechanism of the example connector in a free-spin configuration. FIG. 32 shows a cross-sectional view through the rotation mechanism of the example connector in an engaged configuration.

The connector 2002 can have a proximal end portion 2014 that includes a female luer 2020, external threading 2022, a flange 2024, and an interior lumen 2026. The proximal end portion 2014 can include one or more protrusions or 2027, which can extend radially outward from the outside surface of the interior lumen 2026. Two protrusions or teeth 2027 can be included, such as disposed on opposite sides of the proximal end portion 2014.

The housing 2010 can include a sidewall 2030, a flange 2032 extending inwardly from the sidewall 2030, and an opening 2034 through the flange 2032. The sidewall 2030 can include gaps 2096 (e.g., slits) that can divide the sidewall into four sidewall sections 2030a, 2030b, 2030c, and 2030d. The sidewall sections 2030a and 2030c can be positioned opposite each other. The sidewall sections 2030b and 2030d can be positioned opposite each other. The gaps 2096 can enable the user to move one or more of the sidewall sections 2030a-d between a disengaged configuration and an engaged configuration. For example, the user can press or squeeze opposing sidewall sections 2030b and 2030d inwardly or towards each other, such as by applying force in the direction of the arrows in FIGS. 28 and 32. The user can squeeze the opposing sidewall sections 2030b and 2030d between the user's finger and thumb using one hand. The other hand can hold another connector, such as to twist another connector into engagement with the proximal end portion 2014 of the connector 2002. The housing 2010 can have one or more teeth 2036 formed on the inside of the sidewall 2030. In the embodiments illustrated in FIGS. 27-32, the housing 2010 can have two teeth 2036, but any suitable number (e.g., 2, 3, 4, 6, 8, 10, 12, or more) can be used. The sidewall sections 2030a-d can have teeth formed on opposite sides. When the opposing sidewall sections 2030b and 2030d move inwardly to the engaged configuration, the teeth 2036 on the sidewall sections 2030b and 2030d can engage the teeth 2027 on the proximal end portion 2014, so that rotation of the housing 2010 can be locked against rotation relative to the proximal end portion 2014. When the housing 2010 is in the engaged configuration (e.g., FIGS. 30 and 32), the housing 2010 and proximal end portion 2014 can rotate together, which can enable a user to couple another connector to the proximal end portion 2014 (e.g., by holding the housing 2010 and the other connector).

The user can release the opposing sidewall sections 2030b and 2030d so that they move outwardly to their unflexed or undeformed positions (e.g., the unengaged configuration shown in FIGS. 29 and 31). When in the unengaged configuration the teeth 2036 on the housing 2010 can be spaced outwardly from the teeth 2027 on the proximal end portion 2014. In the disengaged configuration, the teeth 2027 and 2036 do not engage so that the proximal end portion 2014 can rotate in both directions relative to the housing 2010. The disengaged configuration can permit free spinning of the proximal end portion 2014 relative to the housing 2010. In the disengaged configuration, the proximal end portion 2014 can rotate with another connector, which can impede unintended disconnection of the other connector from the proximal end portion 2014. In some cases, the disengaged configuration can impede connection of the other connector to the proximal end portion 2014.

In the embodiment illustrated in FIGS. 27 to 32, the housing has two teeth 2036, where the sidewall section 2030b has one of the teeth 2036 and the opposite side wall section 2030d has the other of the two teeth 2036. The opposing sidewall sections 2030b and 2030d can each have multiple teeth in some embodiments. In some cases, the side wall sections 2030a and 2030c do not have any teeth 2036. Pressing the sidewall sections 2030a and 2030c inwardly would not transition the connector 2002 to the engaged configuration because the sidewall sections 2030a and 2030c do not have teeth 2036 for engaging the teeth 2027 on the proximal end portion 2014. In other configurations, each of the sidewall sections 2030a, 2030b, 2030c, and 2030d can have one or more teeth 2036, so that the connector 2002 can be transitioned to the engaged configuration by pressing either of the opposing sidewall sections 2030a and 2030c or the opposing sidewall sections 2030b and 2030d. In some embodiments, the housing 2010 can include only one moveable section for transitioning the connector 2002 between the engaged and disengaged configurations. For example, two of the gaps 2096 shown in FIGS. 27 to 32 could be omitted. The sidewall 2030 would include a main section that is not configured to move radially, and the sidewall 2030 would include a movable section that is configured to move radially inward to an engaged configuration and radially outward to a disengaged configuration. The movable sidewall section can be separated from the main sidewall section by gaps 2096 (e.g., slits). The movable sidewall section can include one or more teeth 2036 for engaging one or more teeth 2027 on the proximal end portion 2014 when in the engaged configuration. In some embodiments, the connector 2002 can have 2, 4, 6, 8, or more sidewall sections.

In some embodiments, the teeth 2036 can have engagement surfaces and sliding surfaces, and the teeth 2027 can have engagement surfaces and sliding surfaces (e.g., similar to the other embodiments described herein). When the connector is in the engaged configuration, the engagement surfaces of the one or more teeth 2036 can engage with the engagement surfaces of the one or more teeth 2027 to impede rotation of the proximal end portion 2010 relative to the housing 2010 in a tightening direction (e.g., clockwise in FIGS. 31 and 32). When the proximal end portion 2014 is rotated in the loosening direction (e.g., counterclockwise in FIGS. 31 and 32), the sliding surfaces of the one or more teeth 2036 can slide against the sliding surfaces of the one or more teeth 2027 so that the teeth 2027 can rotate past the teeth 2036. In some cases, the opposing sidewall sections 2030b and 2030d can move outwardly (e.g., less movement than returning to the unengaged configuration) to permit the teeth 2027 to rotate past the teeth 2036. The sliding surfaces of the teeth 2027 and/or 2036 can be angled to facilitate rotation of the teeth 2027 past the teeth 2036. In some cases, if a user were to apply sufficient force to press the opposing sidewall sections 2030b and 2030d to the engaged configuration, the sliding surfaces on the teeth 2027 and/or 2036 could bind so that rotation of the proximal end portion 2014 relative to the housing 2010 in the loosening direction would be prevented. In some cases, the user could remove the other connector from the proximal end portion 2014 by squeezing the opposing sidewall sections 2030b and 2030d with sufficient force to lock the proximal end portion 2014 relative to the housing 2010. In some embodiments, the user could grip or manipulate the flange 2024 to hold the proximal end portion 2014 against rotation to disconnect the other connector from the proximal end portion 2014.

In some embodiments, the teeth 2027 and teeth 2036 could be replaced with one or more protrusions that are configured to engage one or more corresponding recesses when the connector is in the engaged configuration (e.g., with the sidewall portions 2030b and 2030d pressed inwardly). Thus, the engaged configuration can impede rotation of the proximal end portion 2014 relative to the housing 2010 in both directions. In the disengaged configuration the one or more protrusions can be retracted from the one or more recesses to permit rotation of the proximal end portion 2014 relative to the housing 2010 in both directions.

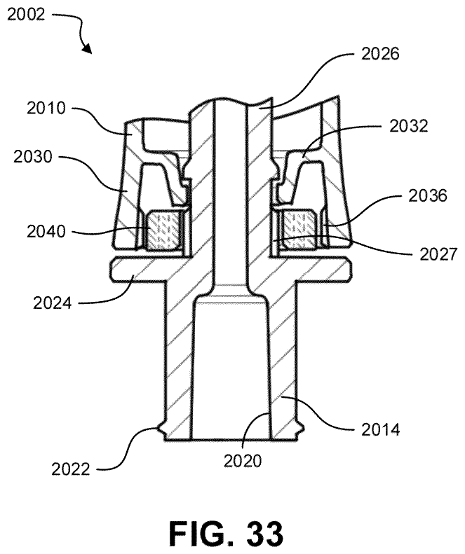

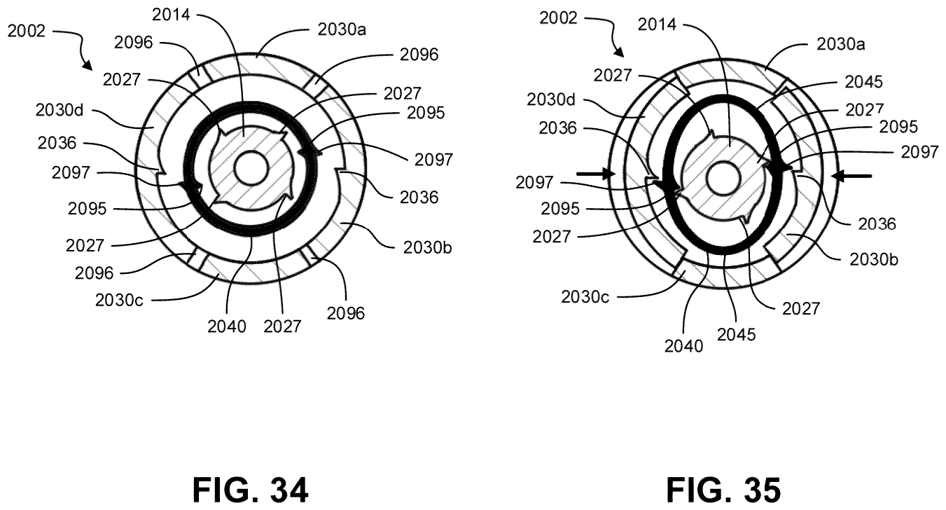

FIG. 33 shows a partial cross-sectional view of the example connector. FIG. 34 shows a cross-sectional view through the rotation mechanism of the example connector in a free-spin or disengaged configuration. FIG. 35 shows a cross-sectional view through the rotation mechanism of the example connector in an engaged configuration. The connector 2002 of FIGS. 33 to 35 can be similar to the embodiments discussed in connection with FIGS. 27 to 32, except as discussed herein. The connector 2002 can include an engagement member 2040, which can have a body portion 2045, which can be annular or circular in shape. The various engagement members 2040 disclosed herein can have other non-circular shapes, such as polygonal shapes. The engagement member 2040 can have one or more inner teeth 2095 disposed on an inside of the body portion 2045 and one or more outer teeth 2097 disposed on an outside of the body portion 2045. The inner teeth 2095 can be configured to engage the one or more teeth 2027 on the proximal end portion 2014 (e.g., when the connector 2002 is in the engaged configuration). The outer teeth 2095 can be configured to engage the one or more teeth 2036 on the housing 2010 (e.g., when the connector 2002 is in the engaged configuration).

The housing 2010 can have four sidewall sections 2030a, 2030b, 2030c, 2030d, although any suitable number of sidewall sections could be used, as discussed herein. Gaps 2096 (e.g., slits) can separate the sidewall sections 2030a-d. One or more of the sidewall sections 2030a-d can be moved to transition the connector between an engaged configuration and a disengaged configuration. For example, opposing sidewall sections 2030b and 2030d can be pressed radially inwardly (e.g., by a force shown by the arrows in FIG. 35) to produce the engaged configuration. When released the opposing sidewall sections 2030b and 2030d can return to their unflexed or undeformed positions (e.g., shown in FIG. 34) to produce the disengaged state.

In the disengaged state (e.g., FIG. 34), the minimum distance between the one or more teeth 2036 on the housing 2010 and the one or more teeth 2027 on the proximal end portion 2014 can provide sufficient space for the engagement member 2040 to rotate between the teeth 2027 and 2036, such as without the inner teeth 2095 engaging the teeth 2027 on the proximal end portion 2014 and/or without the outer teeth 2097 engaging the teeth 2036 on the housing 2010. When in the disengaged state, the proximal end portion 2014 can rotate freely relative to the housing 2010 in both directions. The disengaged configuration and be a free-spin configuration. In some cases, the engagement member 2040 can rotate with the proximal end portion 2014 relative to the housing 2010 (e.g., if the inside teeth 2095 engage with the teeth 2027 on the proximal end portion 2014). In some cases, the engagement member 2040 can rotate with the housing 2010 relative to the proximal end portion 2014 (e.g., if the outside teeth 2097 engage with the teeth 2036 on the housing 2010). In some cases, the housing 2010, the engagement member 2040, and the proximal end portion 2014 can each rotate relative to the other two listed components. The body portion 2045 of the engagement member 2040 can be unflexed or undeformed in the disengaged configuration.

In the engaged state (e.g., FIG. 35), the one or more teeth 2027 on the proximal end portion 2014 can engage the one or more inner teeth 2095 on the engagement member 2040, and the one or more teeth 2036 on the housing 2010 can engage the one or more outer teeth 2097 on the engagement member 2040 to impede rotation of the proximal end portion 2014 relative to the housing 2010 (e.g., in at least the tightening direction). In the engaged configuration, the distance between the sidewall 2030 of the housing 2010 and the internal lumen 2026 of the proximal end portion 2014 can be less than the combined radial lengths of the engagement surfaces on a tooth 2027, tooth 2095, tooth 2097, and tooth 2036. In the engaged configuration, the minimum distance between the teeth 2036 on the housing 2010 and the teeth 2027 on the proximal end portion 2014 can be smaller than the thickness of the engagement member 2040 taken through the teeth 2095 and 2097. In the engaged configuration, rotation of the proximal end portion 2014 in the tightening direction (e.g., clockwise in FIG. 35) can cause the teeth 2027 on the proximal end portion 2014 to engage the inner teeth 2095 on the engagement member 2040. The engagement member 2040 can then rotate with the proximal end portion 2014 in the tightening direction until the outer teeth 2097 engage the teeth 2036 on the housing 2010. In that configuration (e.g., shown in FIG. 35) the proximal end portion 2014 can be impeded from rotating further in the tightening direction relative to the housing 2010. In some embodiments, the body portion 2045 of the engagement member 2040 can be flexed or deformed (e.g., away from the circular or default shape) when the connector 2002 is in the engaged configuration (e.g., FIG. 35). For example, the body portion 2045 can have an oval or oblong annular shape in the engaged configuration. Pressing the sidewall sections 2030b and 2030d inward can press the engagement member 2040 into the deformed shape.

When the one or more sidewall sections 2030a-d are pressed inwardly, the gaps 2096 can be collapsed. The sidewall section 2030a-d being pressed inwardly can abut against one or both adjacent sidewall sections 2030a-d to limit the distance that the sidewall section 2030a-d can be displaced inwardly. In FIG. 35, the opposing sidewall sections 2030b and 2030d are pressed inwardly so that they abut against the ends of the sidewall sections 2030a and 2030c. When the sidewall sections 2030b and 2030d are pressed together, the space between the sidewall sections 2030b and 2030d, or between the teeth 2036 thereon, can permit the engagement member 2040 and/or the proximal end portion 2014 to rotate in the loosening direction. For example, the sliding surface(s) on the housing teeth 2036 and/or on the outer teeth 2097 and/or the sliding surface(s) on the inner teeth 2095 and/or on the teeth 2027 of the proximal end portion 2014 can press against each other and can deform the shape of the engagement member 2040 so that the teeth 2027 can move past the inner teeth 2095 and/or so that the teeth 2036 can move past the outer teeth 2097. By way of example, when the proximal end portion 2014 is rotated in the loosening direction while the sidewall sections 2030b and 2030d are pressed inward, the sliding surface(s) on the one or more teeth 2027 can partially contact the sliding surface(s) on the one or more inner teeth 2095. The engagement member 2040 can rotate in the loosening direction along with the proximal end portion 2014, until the sliding surface(s) of the outer teeth 2097 contact the sliding surface(s) of the teeth 2036 on the housing 2010. As mentioned above, the distance that the sidewall sections 2030b and 2030d can be pressed inward can be limited by stops. When the sidewall sections 2030b and 2030d are pressed together by the full permissible amount, the teeth 2036 can be positioned so that only part of the sliding surfaces of the teeth 2036 contacts only part of the sliding surfaces of the teeth 2097. Additionally or alternatively, the engagement member 2040 can be configured so that only part of the sliding surfaces of the teeth 2095 contact only part of the sliding surfaces of the teeth 2027. This can provide room for the teeth 2095 and 2097 on the engagement member 2040 to be displaced inward (e.g., similar to FIG. 35), so that the outer teeth 2097 can clear the housing teeth 2036. Alternatively, the teeth 2095 and 2097 on the engagement member 2040 can be displaced outward so that the inner teeth 2095 can clear the teeth 2027 on the proximal end portion 2014. In some embodiments, the inner teeth 2095 can be offset from the outer teeth 2097 (e.g., by about 90 degrees, or by about 60 degrees to about 120 degrees, or any values or ranges therein), so that deformation of the engagement member 2040 can cause the inner teeth 2095 to be displaced outward (e.g., to disengage from the teeth 2027) while the outer teeth 2097 are displaced inward (e.g., to disengage from the teeth housing 2036), as the proximal end portion 2014 is rotated in the loosening direction relative to the housing 2010.

The embodiments illustrated in FIGS. 33-35 have four teeth 2027, two inner teeth 2095, two outer teeth 2097, and two teeth 2036, but any suitable number of teeth (e.g., 1, 2, 3, 4, 6, 8, 10, 12, or more) could be used for each set of teeth disclosed. In some embodiments, all of the sidewall sections 2030a-d can have one or more teeth 2036. For example, the connector 2002 can transition to the engaged configuration by squeezing the opposing sidewall portions 2030b and 2030d or by squeezing the opposing sidewall portions 2030a and 2030c, in some configurations. The teeth 2027, 2095, 2097, and/or 2036 can have engagement surfaces and sliding surfaces similar to other embodiments disclosed herein. In some cases, protrusions and corresponding recesses that can be engaged and disengaged can be used in place of some or all of the teeth 2027, 2095, 2097, and 2036, as discussed herein.

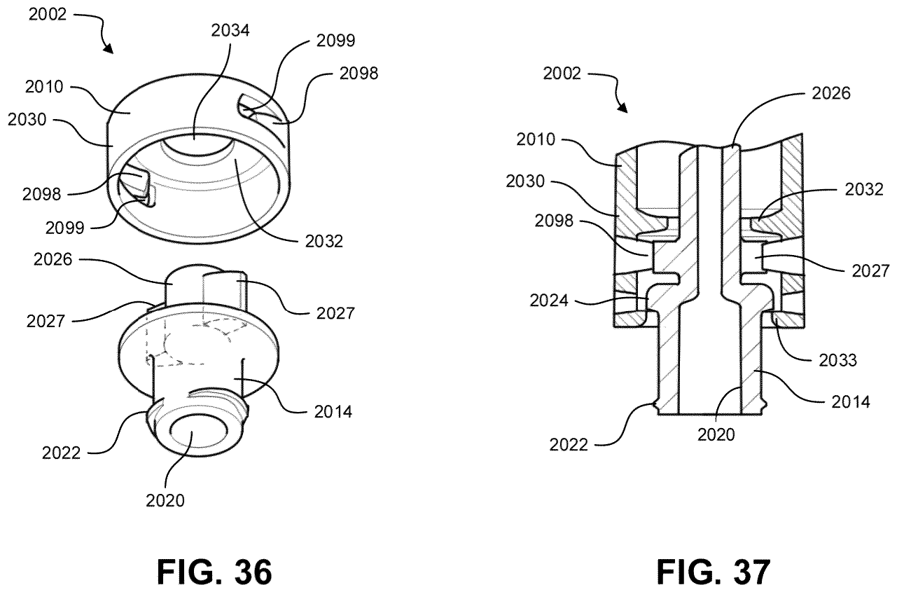

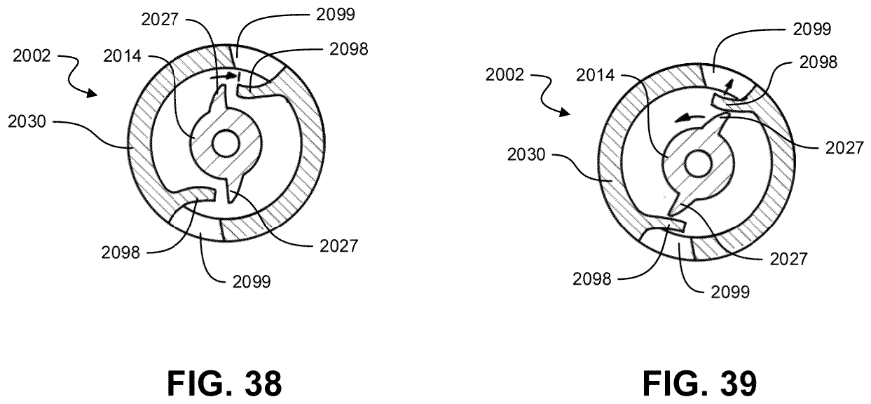

FIG. 36 shows a partial exploded view of another example embodiment of a connector 2002. FIG. 37 shows a partial cross-sectional view of the example connector of FIG. 36. FIG. 38 shows a cross-sectional view through the rotation mechanism of the example connector. FIG. 39 shows another cross-sectional view through the rotation mechanism of the example connector.

The connector 2002 can have a proximal end portion 2014, which can include a female luer 2020, external threading 2022, a flange 2024, an interior lumen 2026, and one or more protrusions or teeth 2027, which can extend radially outward from the interior lumen 2026. The housing 2010 can have a sidewall 2030, a flange 2032 that extends inward from the sidewall 2032, and an opening through the flange 2034. The interior lumen can extend through the opening 2034 in the flange 2032. The housing can have one or more tabs 2033 for securing the proximal end portion 2014 to the housing 2010 (e.g., similar to other embodiments disclosed herein). The one or more tabs 2033 can extend inward from the sidewall 2030, such as at or near the proximal end of the housing 2010. The flange 2024 of the proximal end portion 2014 can be recessed into the housing 2010. The tabs 2033 and/or the flange 2024 can be configured to flex sufficiently to enable the flange 2024 to be inserted distally past the one or more tabs 2033, such as for a snap fit engagement.

The housing 2010 can have one or more pawls 2098, which can extend inwardly from the housing 2010, such as from the sidewall 2030. The pawls 2098 can be configured to engage the teeth 2027 on the proximal end portion 2014. Each of the pawls 2098 can extend from a connection point 2049 on the body 2045. The pawl 2098 can be curved so that an end portion of the pawl 2098 extends along direction generally parallel to the curvature of an adjacent portion of the sidewall 2030. Each of the pawls 2098 can have an engagement surface and a sliding surface. A line normal to the engagement surface can extend generally tangentially to the curvature of the side wall 2030. The sliding surface can be angled so that a line normal to the sliding surface can extend in a direction that is offset from a radially outward direction by an angle of about 5 degrees to about 45 degrees, although other configurations are possible. The side wall can include openings 2099 adjacent to the pawls 2098. The openings 2099 can create space in the sidewall 2030 into which the pawls 2098 can deflect, in some embodiments. In some cases, the openings 2099 can be omitted.

When the proximal end portion 2014 is rotated in a tightening direction (e.g., clockwise in FIG. 38) relative to the housing 2010, such as when coupling another connector to the proximal end portion 2014, the engagement surfaces on the pawls 2098 can engage with engagement surfaces of the teeth 2027 so that the proximal end portion 2014 does not rotate relative to the housing 2010 (e.g., to enable attachment of the other connector to the proximal end portion 2014). When the proximal end portion 2014 is rotated in a loosening direction (e.g., counter clockwise in FIG. 39) relative to the housing 2010, such as when the other connector is turned in a loosening direction, the sliding surfaces of the pawls 2098 can slide against the sliding surfaces of the teeth 2027, and the pawls 2098 can flex outwardly to enable the proximal end portion 2014 to rotate relative to the housing 2010. The force to deflect the pawls 2098 to enable the teeth 2027 of the proximal end portion 2014 to move past the pawls 2098 can be less than the force to twist the other connector for disconnection of the other connector from the proximal end portion 2014 (e.g., similar to other embodiments disclosed herein).

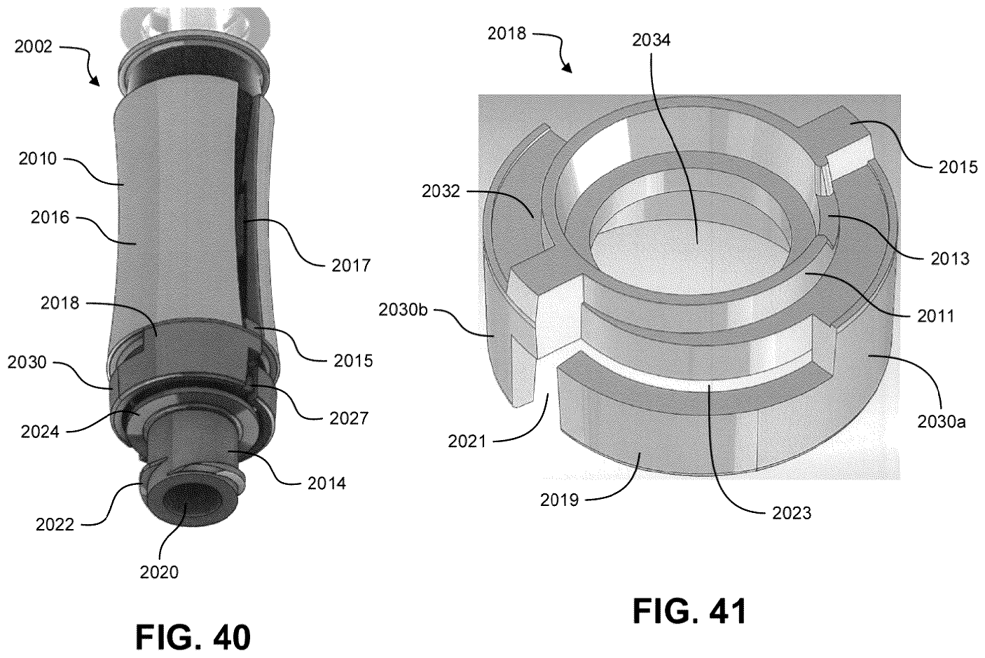

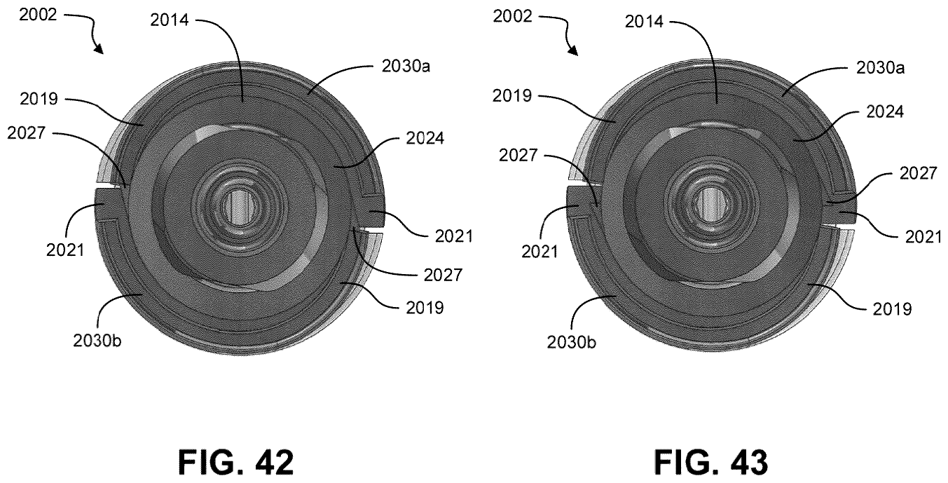

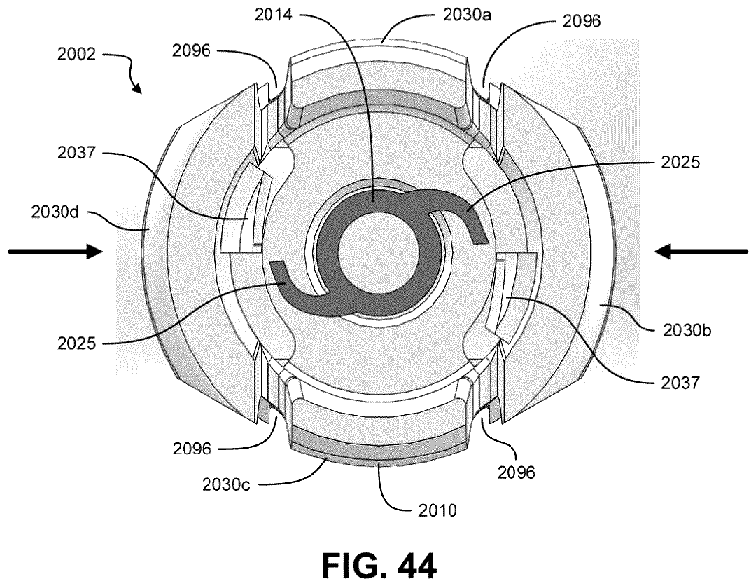

FIG. 40 shows a perspective view of another example embodiment of a connector 2002. FIG. 41 shows proximal housing portion of the connector of FIG. 40. FIG. 42 shows a cross-sectional view of the example connector. FIG. 43 shows another cross-sectional view of the example connector. The connector of FIGS. 40-43 can have features that are the same as or similar to the connector of FIGS. 36-39 (or other embodiments disclosed herein), except as described herein.

The connector 2002 can have a proximal end portion 2014, which includes a female luer 2020, external threading 2022, and a flange 2024. The proximal end portion 2014 can include an interior lumen 2026, which is hidden from view in FIG. 40. The flange 2024 can be disposed proximally of the proximal end of the housing 2010. The flange 2024 can be exposed, in some embodiments, which can enable a user to hold the flange 2024 to restrict rotation of the proximal end portion 2014 (e.g., which can be used to permit removal of another connector from the proximal end portion 2014, as discussed herein). In some embodiments, the flange 2024 can be recessed inside the housing 2010.

The proximal end portion 2014 can include one or more teeth 2027, which can extend radially outward. Each of the teeth can include an engagement surface and a sliding surface. The engagement surface can extend generally radially. A line normal to the engagement surface can extend substantially tangential to a curve along the path of motion of the rotating teeth 2027, or varying from the tangential direction by about 20 degrees, about 15 degrees, about 10 degrees, about 5 degrees, about 3 degrees, about 2 degrees, about 1 degree, or less, or any ranges or values therebetween, although other configurations could be used. The sliding surface can be angled relative to the engagement surface by about 30 degrees, about 35 degrees, about 40 degrees about 45 degrees, about 50 degrees, about 55 degrees, about 60 degrees, about 65 degrees, about 70 degrees, about 75 degrees, or more, or any values or ranges therebetween, although other configurations could be used. The teeth 2027 of FIGS. 40 and 42-43 can be similar to the teeth of other embodiments disclosed herein. In some embodiments, the teeth 2027 can extend radially outward from the interior lumen 2026. In some embodiments, the teeth 2027 can extend radially outward from the flange 2024. The teeth 2027 can extend radially outward past the flange 2024 in some cases.

The connector 2002 can include a housing 2010. The housing can have a distal portion 2016 and a proximal portion 2018, which can be coupled by sonic welding, adhesive, screw threads, pins, snap fit features, friction fit features, or the like. When coupled the distal portion 2016 and the proximal portion 2018 of the housing 2010 can move together and can function as a single unitary component. In some embodiments, the features of the distal portion 2016 and the proximal portion 2018 of the housing 2010 can be formed as a single integrated component. The proximal portion 2018 of the housing can have a sidewall 2030, a flange 2032 that extends inwardly from the sidewall 2030, and an opening 2034 through the flange 2032. Similar to other embodiments disclosed herein, the interior lumen 2026 of the proximal end portion 2014 can extend through the opening 2034 in the flange 2032.

In some embodiments, engagement features can extend upward from the flange 2032 and can be configured, for example, to engage corresponding features on the distal portion 2016 of the housing 2010. For example, a ridge 2011 can extend distally from the flange 2032. The ridge 2011 can have a gap 2013. One or more tabs 2015 can extend distally from the flange 2032 or sidewall 2030. The tabs 2015 can fit into grooves 2017 on the distal portion 2016 of the housing 2010, as shown in FIG. 40 for example.

The housing 2010 can have one or more pawls 2019, which can be configured to interact with the one or more teeth 2027 on the proximal end portion 2014, such as to impede rotation of the proximal end portion 2014 relative to the housing 2010 in a first direction (e.g., a tightening direction that enables another connector to be threaded onto the proximal end portion 2014), and to permit rotation of the proximal end portion 2014 relative to the housing 2010 in a second direction (e.g., a loosening direction so that other connector does not unintentionally detach from the proximal end portion 2014). The embodiments of FIGS. 40 to 43 show two pawls 2019 and two teeth 2027, but any suitable number (e.g., 1, 2, 3, 4, 5, 6, 8, 10, 12, or more) pawl 2019 and/or teeth 2027 could be used.

The pawls 2019 can form part of the sidewall 2030 of the housing 2010. The sidewall 2030 can have a first sidewall section 2030a and a second sidewall section 2030b, which can be spaced apart from each other. The sidewall sections 2030a and 2030b can form part of a generally cylindrical shape. The sidewall sections 2030a and 2030b can be annular segments having a first radius of curvature. Each of the pawls 2019 can extend from one of the sidewall sections 2030a and 2030b and can extend towards a next or opposing sidewall section 2030b and 2030a. The pawls 2019 can have a second radius of curvature that is smaller than the first radius of curvature, such as about 5%, about 10% about 15%, about 20%, about 25% smaller, or any values or ranges therebetween, although configurations could be used. The pawls 2019 can have engagement surfaces. The engagement surface can extend generally radially. A line normal to the engagement surface can extend substantially tangential to a curve along the path of motion of the rotating teeth 2027 (or a curvature path of the pawls 2019 themselves), or varying from the tangential direction by about 20 degrees, about 15 degrees, about 10 degrees, about 5 degrees, about 3 degrees, about 2 degrees, about 1 degree, or less, or any ranges or values therebetween, although other configurations could be used. The engagement surfaces on the pawls 2019 can be at the ends of the pawls 2019. The teeth 2027 on the proximal end portion 2014 can be positioned radially inward of the sidewall sections 2030a and 2030b.

The sidewall sections 2030a and 2030b can be positioned so that the teeth 2027 do not contact or engage the sidewall sections 2030a and 2030b as the teeth 2027 and proximal end portion 2014 rotate relative to the housing 2010. The smaller radius of curvature of the pawls 2019 can cause the ends of the pawls 2019 to extend radially inward further than the sidewall sections 2030a and 2030b. The distance from the longitudinal axis through the connector 2002 to the end of the pawl 2019 can be less than the distance from the longitudinal axis to the sidewall section 2030a or 2030b. As the teeth 2027 rotate with the proximal end portion 2014 the teeth 2027 can contact and engage the pawls 2019, as discussed herein.

Two sidewall sections 2030a and 2030b are shown, but a different number of sidewall sections could be used, such as if the connector 2002 included a different number of pawls. Each sidewall section 2030a, 2030b can have a corresponding pawl 2019 extending therefrom. In some embodiments, gaps 2021 can be disposed between the ends of the pawls 2019 and the ends of the sidewall sections 2030a, 2030b. The flange 2031 can extend inwardly from the sidewall sections 2030a and 2030b. The flange 20301 can connect the sidewall sections 2030a and 2030b. In some embodiments, the pawls 2019 are not directly connected to the flange 2032. A gap 2023 can be disposed between the pawl 2019 and the flange 2032. The pawl 2019 can be moveable (e.g., to flex outwardly). The pawls 2019 can have sliding surfaces, which can be the radially inwardly facing sides of the pawls 2019, which can be configured to slide relative to the teeth 2027, as described herein.

When the proximal end portion 2014 is rotated in a second or tightening direction (e.g., clockwise in FIG. 42), the engagement surfaces of the teeth 2017 can abut against the engagement surfaces of the pawls 2019 (e.g., as shown in FIG. 42). The respective engagement surfaces can be configured to be substantially coplanar when they abut against each other, and the engagement surfaces can be substantially orthogonal to the direction of the force that presses them together. The engagement between the teeth 2027 and the pawls 2019 can impede the proximal end portion 2014 from rotating further in the tightening direction relative to the housing 2010. This configuration can enable a user to attach another connector onto the proximal end portion 2014, such as by holding the housing 2010 and the other connector and rotating them relative to each other in a tightening direction.

When the proximal end portion 2014 is rotated in a first or loosening direction (e.g., counterclockwise in FIG. 43), the engagement surface of the tooth 2027 can move away from the corresponding engagement surface of the pawl 2019. Rotation of the proximal end portion 2014 in the loosening direction further than the position shown in FIG. 43 can cause the teeth 2027 to contact the sliding surfaces on the pawls 2019. Sliding surfaces or tips of the teeth 2027 can slide along the radially inward sides of the pawls 2019, thereby causing the pawls 2019 to flex radially outward. Once the teeth 2027 move past the ends of the pawls 2019, the pawls can return to their unflexed positions (e.g., with a snap or click). The proximal end portion 2014 can rotate freely in the loosening direction relative to the housing 2010. If the other connector that is attached to the proximal end portion 2014 is rotated in the loosening direction, it can rotate the proximal end portion 2014 in the loosening direction as well, which can prevent disconnection of the other connector form the proximal end portion 2014. The threshold force to slide the teeth 2017 along and past the pawls 2019 can be less than the threshold force to unthread or otherwise uncouple the other connector from the proximal end portion 2014.

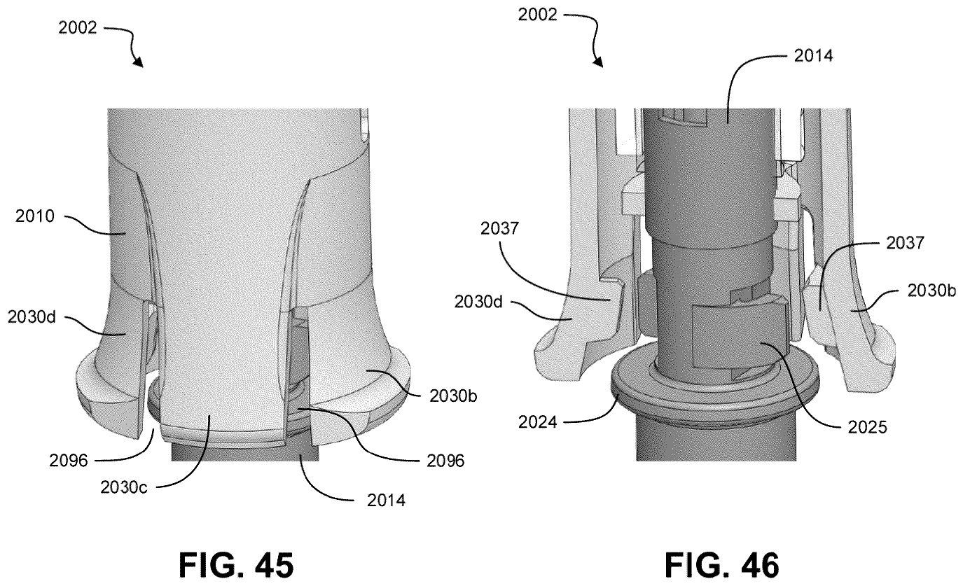

FIG. 44 shows a cross-sectional view of another example embodiment of a connector 2002, which can be similar to the embodiments of FIGS. 27 to 32, except as discussed herein. FIG. 45 is a partial perspective view of the example connector 2002 of FIG. 44. FIG. 46 is a partial perspective view of the example connector 2002 of FIG. 44 with a portion of the housing 2010 cut away to facilitate illustration of the interior of the connector 2002. The connector 2002 can have a proximal end portion 2014, which can be coupled to a housing 2010 so that the proximal end portion 2014 can rotate relative to the housing 2010 (e.g., about a longitudinal axis of the connector 2002). The housing 2010 can include multiple sidewall sections 2030a-d separated by gaps 2096 (e.g., similar to any of FIGS. 27-35). For example, the housing 2010 can have four sidewall sections 2030a-d, although any suitable number of sidewall sections (e.g., 2, 4, 6, 8, or more) can be used. Opposing sidewall sections 2030b and 2030d can be pressed inwardly to transition the connector from a disengaged configuration or state to an engaged configuration or state. When released, the opposing sidewall sections 2030b and 2030d can return to the unengaged state.

The opposing sidewall sections 2030b and 2030d can have one or more protrusions 2037, which can extend inwardly from the sidewall sections 2030b and 2030d. The protrusions 2037 can be similar to the housing teeth 2036 disclosed herein. In some embodiments, the projections 2037 can include an engagement surface, which can be similar to other embodiments disclosed herein. A line normal to the engagement surface can extend substantially tangential to a curve along the path of motion of the protrusions 2037, or varying from the tangential direction by about 20 degrees, about 15 degrees, about 10 degrees, about 5 degrees, about 3 degrees, about 2 degrees, about 1 degree, or less, or any ranges or values therebetween, although other configurations could be used.

The proximal end portion 2014 can include one or more pawls 2025, which can be configured to engage the protrusions 2037 when the connector is in the engaged state (e.g., with the sidewall sections 2030b and 2030d pressed inward). The pawls 2025 can be similar to other embodiments disclosed herein. The pawls 2025 can extend out from a connection point on the proximal end portion 2014 (e.g., from an interior lumen 2026 thereof). The pawl 2025 can be curved so that an end portion of the pawl 2025 extends along a direction that is generally tangential to a curve along the path of motion of the rotating pawls 2025. The pawls 2098 can have an engagement surface and a sliding surface, similar to other embodiments discussed herein. A line normal to the engagement surface can extend generally tangentially to a curve along the path of motion of the engagement surface as the pawls 2025 rotate relative to the housing 2010, or varying from the tangential direction by about 20 degrees, about 15 degrees, about 10 degrees, about 5 degrees, about 3 degrees, about 2 degrees, about 1 degree, or less, or any ranges or values therebetween, although other configurations could be used.

When the connector 2002 is in the engaged configuration, rotation of the proximal end portion 2014 in the tightening direction (e.g., clockwise in FIG. 44) relative to the housing 2010 can cause the engagement surface(s) of the one or more pawls 2025 to abut against the engagement surface(s) of the one or more protrusions 2037 to impede further rotation of the proximal end portion 2014 in the tightening direction relative to the housing 2010. In that configuration, further rotation of the proximal end portion 2014 would cause the housing 2010 to rotate with the proximal end portion 2014. This configuration can enable a user to attach another connector to the proximal end portion 2014 by twisting the other connector in the tightening direction while holding the housing 2010, as discussed herein.

When the connector 2002 is in the engaged configuration, rotation of the proximal end portion 2014 in the loosening direction (e.g., counter clockwise in FIG. 44) relative to the housing 2010 can cause the pawl(s) 2025 to contact the protrusion(s) so that the pawl(s) are deflected to permit the pawl(s) 2025 to rotate past the corresponding protrusion(s). For example, the pawl 2025 can have a sliding surface that is configured to contact the protrusion 2037 and slide along the protrusion 2037 as the proximal end portion 2014 rotates in the loosening direction. The pawl 2025 can be deflected (e.g., inwardly) when the sliding surface of the pawl 2025 engages the protrusion 2037. When the pawl 2025 rotates past the protrusion 2037, the pawl 2025 can return to its default or unflexed position (e.g., shown in FIG. 44). The force that causes the one or more pawls 2025 to deform and rotate past the one or more protrusions 2037 can be less than the force to detach another connector that is coupled to the proximal end portion 2014. Thus, if a user holds the housing 2010 and rotates the other connector in the loosening direction, the proximal end portion 2014 will merely rotate with the other connector in the loosening direction relative to the housing 2010, thereby impeding disconnection of the other connector from the proximal end portion 2014. Thus, the connector 2002 can be configured to impede decoupling of another connector from the proximal end portion even when the connector 2002 is in the engaged configuration.

The distance that the sidewall sections 2030b and 2030d can be pressed inward can be limited by stops, similar to other embodiments disclosed herein. For example, when the second sidewall section 2030b is pressed inward, a first end of the second sidewall section 2030b can abut against a first end of the first sidewall section 2030a and/or a second end of the second sidewall section 2030b can abut against a first end of the third sidewall section 2030c. When the fourth sidewall section 2030d is pressed inward, a first end of the fourth sidewall section 2030d can abut against a second end of the first sidewall section 2030a and/or a second end of the fourth sidewall section 2030d can abut against a second end of the third sidewall section 2030c. Displacing the one or more sidewall sections 2030a-d to the engaged configuration can collapse one or more corresponding gaps 2096. The size of the corresponding gaps 2096 can set the displacement distance that the one or more sidewall sections 2030a-d can be displaced. When the one or more corresponding gaps 2096 are collapsed, the one or more sidewall sections 2030a-d can be stopped from being displaced further inward (e.g., even if additional squeezing force were applied).

The stops can impede the protrusions 2037 from being displaced so far inward that they would block the pawls 2025 and proximal end portion 2014 from rotating in the loosening direction relative to the housing 2010. For example, when the sidewall sections 2030b and 2030d are pressed inward until they abut against the other sidewall sections 2030a and 2030c, the protrusions 2037 can be positioned so that they can displace the pawls 2025 inward to permit the proximal end portion 2014 to rotate in the loosening direction (e.g., counter clockwise in FIG. 44), and so that they can engage the pawls 2025 to impede rotation of the proximal end portion 2014 in the tightening direction (e.g., clockwise in FIG. 44).

When the connector 2002 is in the disengaged configuration (e.g., as shown in FIG. 44), the one or more protrusion(s) 2037 can be positioned to not engage, or to not contact, the pawls 2025 as the proximal end portion 2014 rotates relative to the housing 2010. In the disengaged state, the connector 2002 can permit the proximal end portion 2014 to spin freely in both directions relative to the housing 2010. This feature can be beneficial to avoid twisting or tangling a fluid line coupled to the connector 2002. This feature can be beneficial to avoid unintended disconnection of the connector 2002 from another connector. A threshold force to rotate the proximal end portion 2014 in the loosening direction can be lower when the connector 2002 is in the disengaged state than when the connector 2002 is in the engaged state, even though both states permit rotation of the proximal end portion 2014 in the loosening direction relative to the housing. This can be because the pawls 2025 do not contact the protrusions 2037 when the proximal end portion 2014 rotates relative to the housing 2010 with the connector 2002 in the disengaged state, whereas the pawls 2025 are deflected by the protrusions 2037 to permit rotation of the proximal end portion 2014 in the loosening direction when the connector 2002 is in the engaged state.

Many variations are possible. For example, the sidewall sections 2030b and 2030d, or any other suitable portion of the housing 2010, can have one or more flexible pawls 2037 instead of the protrusions 2037. The proximal end portion 2025 can have relatively inflexible protrusions (e.g., similar to protrusions 2037) instead of the pawls 2025. In some embodiments, both the housing 2010 and the proximal end portion 2014 can include flexible pawls (e.g., similar to the pawls 2025, but facing in opposite directions).

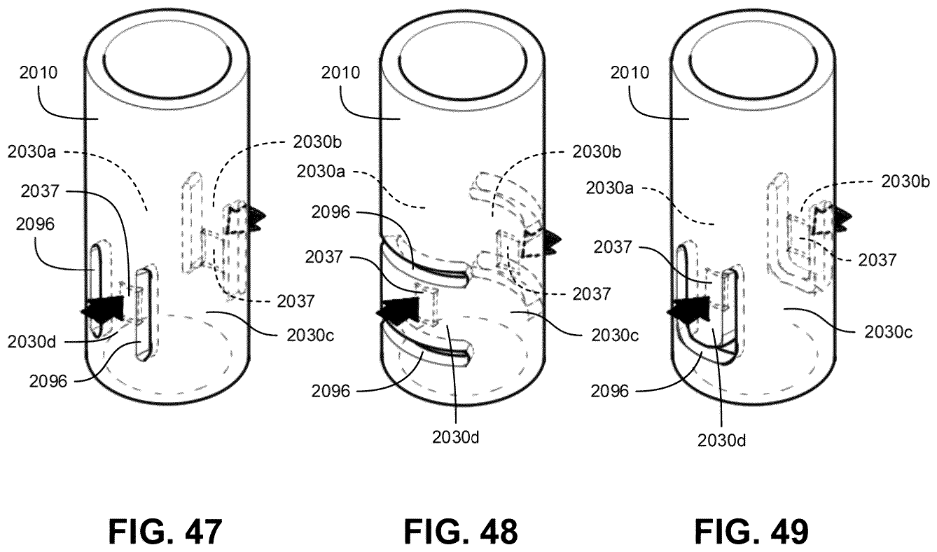

In some embodiments, the gaps 2096 between the sidewall sections 2030a-d can extend to the end of the housing 2010, such as to form open slits, as shown for example in FIG. 45. In some implementations, the gaps 2096 do not extend to the end of the housing 2010, and the gaps 2096 can be enclosed, as shown for example in FIG. 47, which shows an example housing 2010 for a connector 2002. The gaps 2096 can be closed slits, which can have an elongate shape that extends generally longitudinally along the connector 2002. The embodiment of FIG. 47 can be similar to other embodiments disclosed herein, such as FIGS. 44-46, except that the gaps 2096 are spaced away from the end of the housing 2010. The inside surface of the housing 2010 can have one or more protrusions 2037 which can be moved inward to engage one or more pawls 2025 or other features to limit rotation of the housing 2010 relative to the inner structure or fluid pathway, similar to other embodiments disclosed herein. The housing 2010 can have one or more sidewall sections 2030b, 2030d that have the one or more protrusions 2037. The housing 2010 can have one or more sidewall sections 2030a, 2030c, which do not have the protrusions 2037 in some embodiments. The gaps 2096 can separate the one or more sidewall sections 2030b, 2030d from the other one or more sidewall sections 2030a, 2030c. The gaps 2096 can facilitate inward motion of the one or more sidewall sections 2030b, 2030d, such as when a user pinches them inwardly. The housing 2010 can have two projections 2037 position on opposing sides, but other configurations are possible, such as with a single projection 2037, or 4 projections, etc. The gaps 2096 can be replaced with flexible sections.

FIG. 48 shows another example housing 2010 for a connector 2002, which can be similar to the embodiment of FIG. 47 or the other embodiments disclosed herein. In the embodiment of FIG. 48, the gaps 2096 extend laterally. The gaps 2096 can provide one or more movable sections 2030b, 2030d of the housing 2010 that can be pressed inward by a user squeezing or pinching the one or more movable sections 2030a, 2030b of the housing 2010. For example, movable sections 2030b, 2030d can be disposed between the gaps 2096. In the example of FIG. 48, the sidewall sections 2030b and/or 2030d can be a movable portion of the housing 2010, which can be displaced inwardly to provide the engaged configuration similar to other embodiments disclosed herein. In some embodiments, the housing 2010 can have a single movable section 2030d, which can be used to transition between the engaged and disengaged configurations, as discussed, although any suitable number of movable sections could be used. The gaps 2096 can extend along a direction that is substantially parallel with the longitudinal axis (e.g., as shown in FIG. 47), along a direction or on a plane that is substantially perpendicular to the longitudinal axis (e.g., as shown in FIG. 48), or various other suitable directions to facilitate movement of one or more housing sections 2030b, 2030d.

FIG. 49 shows an example embodiment of a housing 2010, which can be used for a connector 2002. The example of FIG. 49 can be similar to the examples of FIGS. 47 and 48, or other embodiments disclosed herein. In the example of FIG. 49, a single continuous gap 2096 can extend around enough of the movable housing portion 2030b or 2030d to facilitate movement of the housing portion 2030b or 2030d when pressed inward by the user. The example of FIG. 49 can be similar to the example of FIG. 47, except that the ends of the two gaps 2096 can be connected by a lateral gap section to form a single continuous gap 2096. The gap 2096 can surround a majority of the corresponding movable housing portion 2030b or 2030d, such as surrounding about 60%, about 65%, about 70%, about 75%, about 80%, about 85%, about 90% or more of the corresponding movable housing portion 2030b or 2030d, or any values or ranges between any of these values.

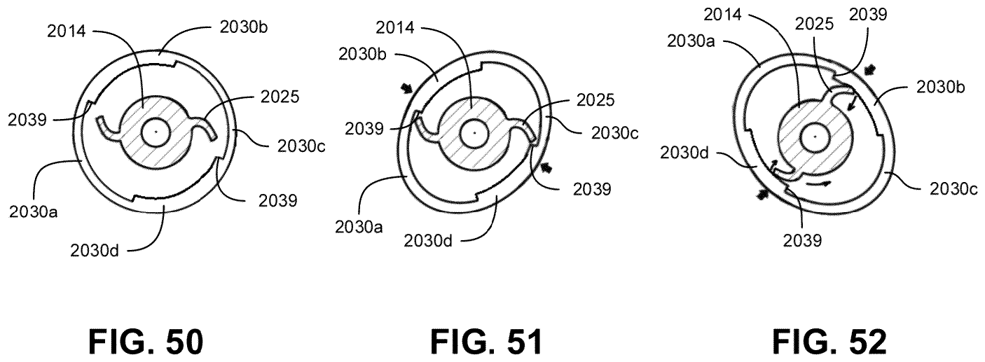

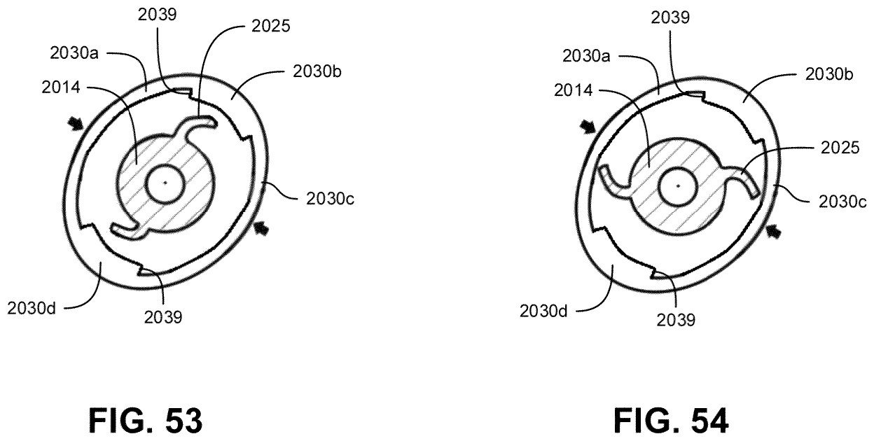

In some embodiments, the gaps 2096 can be omitted. The one or more movable sidewall sections 2030b and 2030d can be defined by different thicknesses of the sidewall 2030. By way of example, the sidewall sections 2030a and 2030c can be thinner than the sidewall sections 2030b and 2030d, as shown in FIG. 50. Pressing the sidewall sections 2030b and 2030d together as shown in FIG. 51 can cause the thinner sidewall sections 2030a and 2030c to flex so that the sidewall sections 2030b and 2030d can be displaced towards each other. Steps 2039 can be formed at transitions between the sidewall sections 2030a and 2030d and between the sidewall sections 2030b and 2030c. The steps 2039 can provide engagement surfaces that can engage the engagement surfaces on the pawls 2025 to impede rotation of the proximal end portion 2014 in the tightening direction (e.g., clockwise in FIGS. 50 to 52). FIG. 50 shows a disengaged configuration, which can be the default or undeformed configuration, where the steps 2039 can be positioned radially outward of the rotational path of the pawls 2025. In the disengaged configuration, the proximal end portion 2014 can rotate freely in both directions relative to the housing 2010. FIG. 51 shows an engaged configuration, where an external force (e.g., a user pinching the housing 2010) displaces the side wall sections 2030b and 2030d inwardly so that at least one engagement feature (e.g., the steps 2039) is disposed in the rotational path of the pawls 2025. The step 2039 or transition from the thinner sidewall section to the thicker sidewall section can provide a catch point that engages the pawl(s) 2025. As can be seen in FIG. 50, in the engaged configuration, rotation of the proximal end portion 2014 in the tightening direction can cause the pawls to abut against the steps 2039 to impede further rotation of the proximal end portion 2014 in the tightening direction. However, rotation of the proximal end portion 2014 in the loosening direction (e.g., counter-clockwise in FIG. 52), can cause pawls 2025 to be displaced inwardly and slide across the corresponding sidewall sections 2030b and 2030d, thereby permitting the proximal end portion 2014 to continuously rotate in the loosening direction, even in the engaged configuration. As shown in FIGS. 53 and 54, in some embodiments, applying force that presses the thinner sidewall sections 2030a and 2030c towards each other would not transition the connector 2002 to the engaged configuration. In some cases, that force can displace the steps 2039 further away from the rotational path of the pawls 2025.

Various engagement features could be used, such as the protrusions, pawls, teeth, and recesses disclosed herein. Generally, where suitable, engagement features of one type can be used in place of engagement features of another type, even when not specifically shown. For example, in some instances, pawls that engage teeth could be replaced with teeth that engage pawls, or pawls that engage recesses, or protrusions that engage recesses, etc. In some cases, one or more recesses can be configured to engage one or more corresponding pawls, teeth, or protrusions. In some cases, a recess can have an engagement surface at one end (e.g., to limit rotation of a corresponding engagement feature) and/or an angled sliding surface at another end (e.g., to permit rotation of a corresponding engagement feature). In some cases, pawl(s) can press outward against a surface (e.g., sidewall) as the pawls and sidewall rotate relative to each other, and the surface can have recess(es) that permit the pawl(s) to move to a less flexed position to engage the recess(es) to impede relative rotation.

In some embodiments, the engagement features (e.g., one or more pawls, teeth, or protrusions) can be integrally formed with the corresponding component of the connector 2002 (e.g., the housing 2010 or the proximal end portion 2014). Alternatively, the engagement features (e.g., one or more pawls, teeth, or protrusions) can be separately formed and can be coupled to move (e.g., rotate) with the corresponding component of the connector 2002. For example, in FIG. 44, the pawls 2025 could be integrally formed with the proximal end portion 2014 as shown, or the pawls 2025 could be part of an engagement member 2040 that is keyed to rotate with the proximal end portion 2014, similar to the engagement member 2040 of FIG. 6. In some embodiments, features that move with a component, such as the housing 2010 or proximal end portion 2014, can be considered to be part of that component even if they are separately formed. In some cases, engagement features on the components (e.g., on the housing 2010 and the proximal end portion 2014) can engage each other by direct contact, while in other embodiments, the engagement features can have indirect engagement, such as through an engagement member 2040, as disclosed herein.

In some disclosed embodiments, engagement features can extend outward from the outside surface of an interior lumen 2026 of the proximal end portion 2014. However, engagement features could extend from, or be disposed on, other portions of the proximal end portion 2014, such as from the flange 2024, or from the barrel of the female luer fitting, or from another component that rotates or otherwise moves with the proximal end portion 2014.

The rotation mechanisms disclosed herein can be incorporated into various types of connectors. For example, the rotation mechanisms can be used in a connector similar to the connector 100 of the ′712 Publication, such as to control rotation between the male housing 123 and the first cap component 132. The rotation mechanisms can be used in a connector similar to the connector 400 of the ′712 Publication, such as to control rotation between the female housing 440 and the fluid conduit 280. The rotation mechanisms can be used in a connector similar to any of the connectors 1100, 2100, 3100, 8100, 9100 of the ′712 Publication, such as to control rotation between the male housing 1123, 2123, 3123, 8123, 9123 and the first cap component 1132, 8132, 9132. The rotation mechanisms can be used in a connector similar to any of the connectors 1400, 2400, 8400 of the ′712 Publication, such as to control rotation between the female housing 1440, 8440, and the first cap component 1420. The rotation mechanisms disclosed herein can be used in connection with various other connectors disclosed in the ′712 Publication or otherwise, such as to control rotation of a first portion relative to a second portion. Any suitable features of the connectors disclosed in the ′712 Publication can be incorporated into the connector 2002 embodiments disclosed herein. In some embodiments, the proximal end distal ends can be interchanged, and the proximal end portion 2014 can be a distal end portion. Although various embodiments are discussed in connection with a proximal end portion, a distal end portion, end portion, connector fitting portion, or other component can be rotatable relative to the housing using the rotation mechanisms disclosed herein.

In some embodiments, the male and female luer fittings can be exchanged. For example, the female luer fitting can be part of, or movable with, the housing, and the male luer fitting can be part of, or movable with, a different component that can rotate relative to the housing. Although some embodiments disclose luer lock fittings, other types of connection fittings could be used.

The proximal end portion (or first portion) of the connector can be configured to couple to a first medical implement, such as a syringe, fluid line, IV bag, or any other suitable medical device. The distal end portion (or second portion) of the connector can be configured to couple to a second medical implement, such as a catheter, fluid line, IV bag, or any other suitable medical device. The connector 2002 can be configured to transfer fluid between the first and second medical implements (e.g., for infusing fluid through the connector 2002 and into a patient). The rotation mechanisms disclosed herein can control rotation between the proximal end portion (or first portion) and the distal end portion (or second portion) of the connector 2002.

Some embodiments can permit rotation of a first portion of the connector (e.g., the proximal end portion 2014) relative to a second portion of the connector (e.g., the housing 2010), which can allow continuous rotation, such as repeated 360-degree revolutions. Some embodiments can impede rotation of a first portion of the connector (e.g., the proximal end portion 2014) relative to a second portion of the connector (e.g., the housing 2010) in one or more directions. In some instances, the first portion of the connector can rotate relative to the second portion of the connector by a limited range before the rotation is impeded. For example, a proximal end portion 2014 may be permitted to rotate in a second direction (e.g., a tightening direction) relative to the housing 2010 for a limited distance until the engagement features of the rotation mechanism engage to impede further rotation in the second direction.

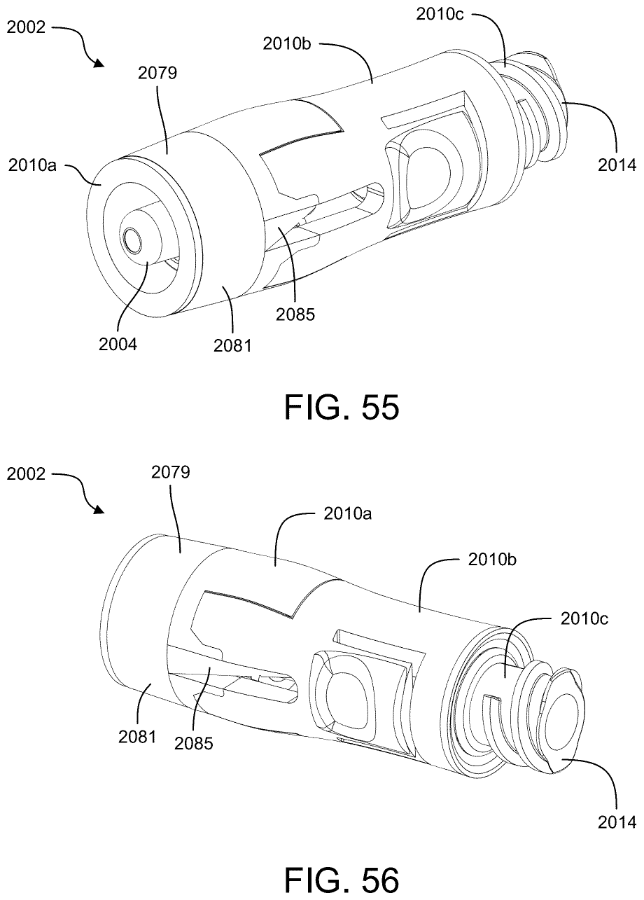

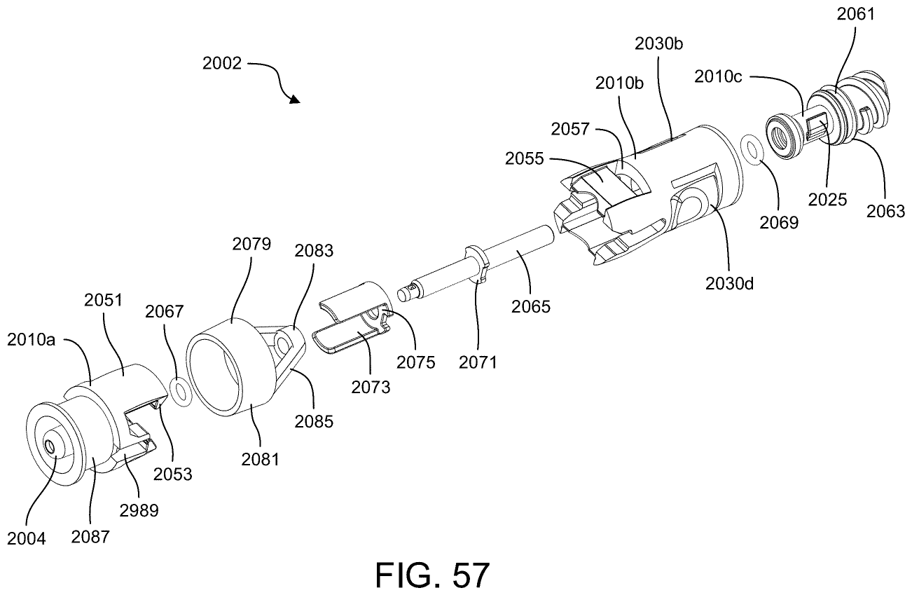

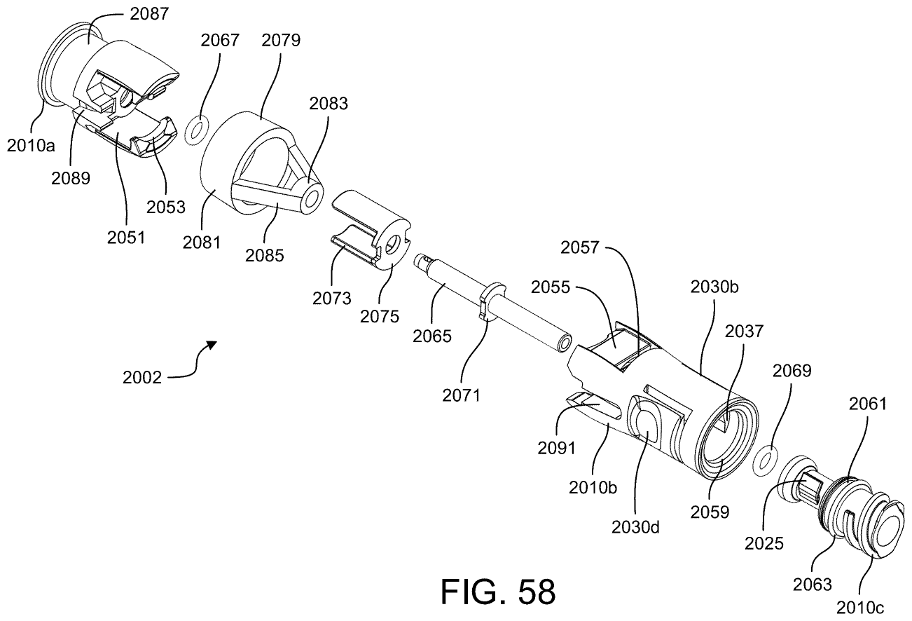

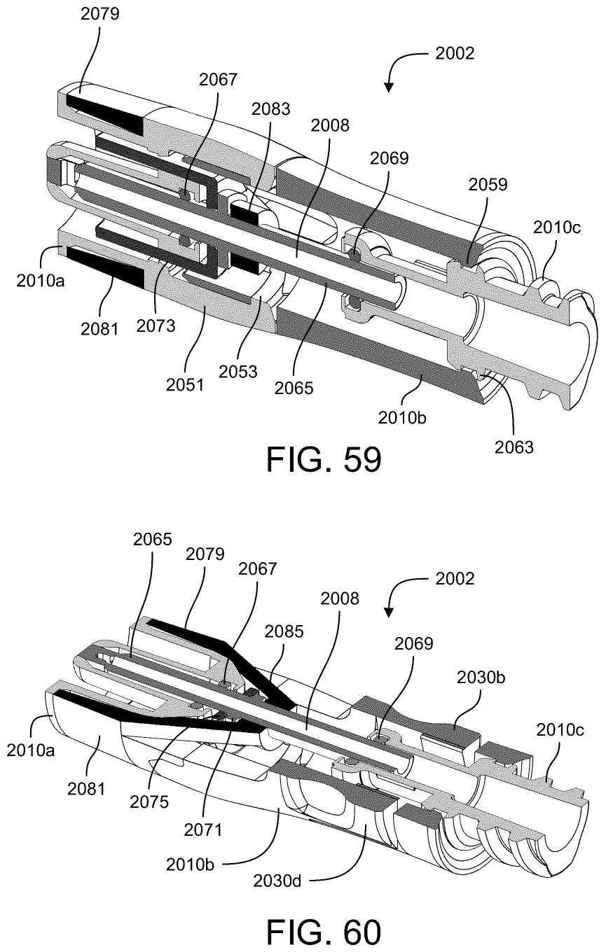

FIG. 55 is a perspective view of an example embodiment of a connector 2002. In some cases, the example connector 2002 can include features similar to those of other embodiments disclosed herein. FIG. 56 is another perspective view of the example connector 2002. FIG. 57 is an exploded view of the example connector 2002. FIG. 58 is another exploded view of the example connector 2002. FIG. 59 is a cross-sectional view of the example connector 2002. FIG. 60 is another cross-sectional view of the example connector 2002.

The connector 2002 can have a housing 2010, which can include multiple housing portions, in some cases. For example, the connector 2002 can include a first or distal housing portion 2010a, a second or central housing portion 2010b, and a third or proximal housing portion 2010c. The portions of the housing 2010 can be coupled together, such as by snap fit features, although in some cases sonic welding, adhesive, screw threads, pins, friction fit features, or other coupling features can be used. The first housing portion 2010a can have one or more arms 2051 with protrusions 2053 (e.g., two opposing arms 2051). The one or more arms 2051 can extend into corresponding one or more slots 2055 on the second housing portion 2010b (e.g., two opposing slots 2055). The exterior of the arms 2051 can be substantially flush with the exterior of the second housing portion 2010b adjacent to the slots 2055. The one or more protrusions 2053 can engage one or more corresponding recesses or openings 2057 on the second housing portion 2010b (e.g., two opposing recesses or openings 2057) to couple the first housing portion 2010a to the second housing portion 2010b, such as with a snap-fit engagement. Various alternatives are possible. For example, in some cases, the first housing portion 2010a can have one or more recesses or openings that receive one or more protrusions on second housing portion 2010b, or other engagement features can be used.

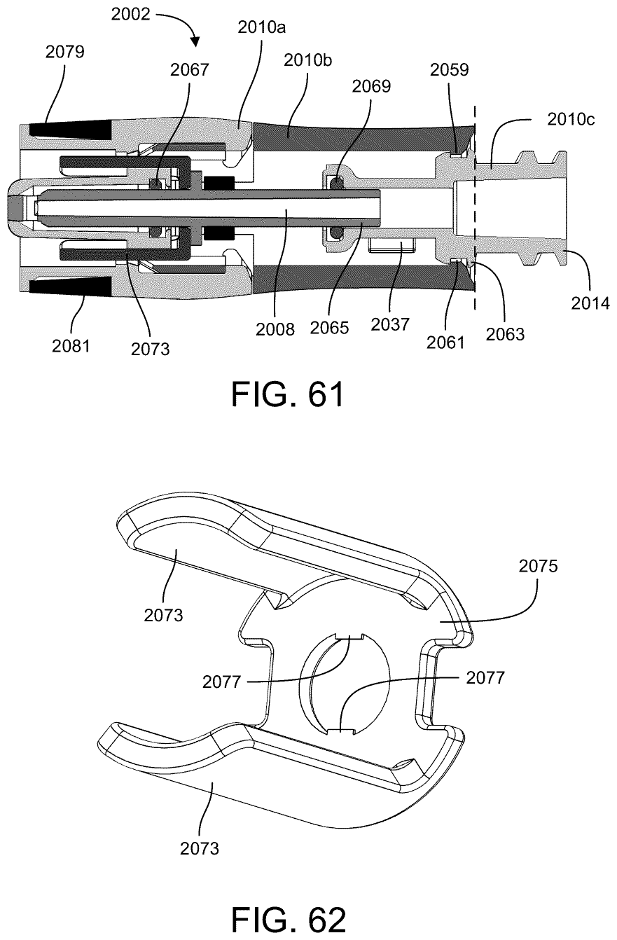

The second housing portion 2010b can have a ridge or protrusion(s) 2059 that can extend inward to engage a corresponding channel 2061 on the third housing portion 2010c, which can couple the third housing portion 2010c to the second housing portion 2010b. The ridge or protrusion(s) 2059 can slide within the channel 2061 to permit the third housing portion 2010c to rotate relative to the second housing portion 2010b (e.g., about a longitudinal axis). Various alternatives are possible. For example, in some cases, the third housing portion 2010c can have the protrusion(s) and the second housing portion 2010b can include the channel, or other engagement features can be used. The channel 2061 can be formed between a distal wall and a proximal wall 2063. The ridge or protrusion(s) 2059 can be recessed distally from the proximal end of the second housing portion 2010b, for example so that the proximal wall 2063 is disposed at least partially inside of the second housing portion 2010b, as can be seen in FIG. 61, which includes a dashed line marking the proximal end of the second housing portion 2010b. In some cases, a generally proximal-facing surface of the second housing portion 2010b can be angled so as to provide a recess, which can receive at least a portion of the proximal wall 2063. In some cases, the proximal wall 2063 can be fully disposed within the recessed portion of the second housing portion 2010b. In some cases, a majority of the proximal wall 2063 can be recessed within the second housing portion 2010b. In embodiments where the third housing portion 2010c includes other engagement features, those engagement features can be at least partially recessed within the second housing portion 2010b, or a majority of those engagement features can be recessed within the second housing portion 2010b, or the entire engagement features can be recessed within the second housing portion 2010b. Having the engagement features recessed within the second housing portion 2010b can impede those engagement features from being gripped by a user or from being accidentally moved in a way that could cause the third housing portion 2010c to rotate and loosen or disconnect from another connector or medical device that is coupled to the third housing portion 2010c (e.g., to the female luer). Thus, the recessed engagement features can facilitate locking the connector 2002 onto another medical device, as discussed herein.

The housing 2010 (e.g., the first housing portion 2010a) can include a distal end 2004, which can include a distal opening. The housing 2010 (e.g., the first housing portion 2010a) can include a tapered male luer, although various other connection types can be used. The first housing portion 2010a can include a hollow projection. The male luer or hollow projection can be surrounded by a shroud, which can have internal threading, such as to engage threading or protrusion(s) on a corresponding locking female luer connector. The internal threading can be similar to the example connector of FIG. 2. The shroud can be part of the housing 2010 (e.g., the first housing portion 2010a).

The housing 2010 (e.g., the third housing portion 2010a) can include a proximal end 2014. The housing 2010 (e.g., the third housing portion 2010a) can include a tapered female luer, although other types of connections can be used. The housing 2010 (e.g., the third housing portion 2010a) can have external threading or protrusions, such as to engage a locking male luer of another connector. The proximal end 2014 can have a proximal opening, such as at the end of the female luer. Many variations are possible. The housing 2010 can include fewer or more housing portions than shown in the example illustrations. In some cases, the first housing portion 2010a and the second housing portion 2010b can be combined into a single housing portion. Accordingly, in some cases, the third housing portion 2010c can be a second housing portion that is coupled to a first housing portion, which can be similar to the housing portions 2010a and 2010b.

A fluid pathway 2008 can extend between the distal end 2004 (e.g., the first or distal opening) and the proximal end 2014 (e.g., the second or proximal opening) of the connector 2002, such as to enable fluid to be transferred through the connector 2002. In some embodiments, the fluid pathway within the connector 2002 may be continuous or without interruption between the distal end 2004 and proximal end 2014. The connector 2002 can have a closure system 2006, which can include a valve or seal. The closure system 2006 can have an open configuration, which can enable fluid to flow through the fluid pathway 2008. The closure system 2006 can have a closed configuration, which can impede fluid from flowing through the fluid pathway 2008. For example, the closure system 2006 can close the distal end 2004 (e.g, the distal opening) of the connector 2002, in some embodiments, although any suitable closure system can be used. In some embodiments, the closure system 2006 can automatically transition to the open configuration when a corresponding connector is attached to the connector 2002 (e.g., to the distal end 2004), as discussed herein. The closure system 2006 can automatically transition to the closed configuration when a corresponding connector is detached from the connector 2002 (e.g., from the distal end 2004), as discussed herein.

The connector 2002 can include a valve member 2065. The valve member can move axially between a closed configuration or distal position, which can seal an inside of the male luer, and an open configuration or proximal position, which can open the male luer. The valve member 2065 can have an opening at its proximal end, and a channel can extend from the opening toward the distal end of the valve member 2065 (e.g., along a longitudinal or axial direction). The distal end of the valve member 2065 can be closed. For example, in some cases, the channel does not extend the full length of the valve member 2065. One or more lateral openings through the side of the valve member 2065 can provide access to the fluid pathway 2008 when the valve member 2065 is in the open configuration. A cross-channel can extend laterally through the valve member 2065 to form two opposing opendings. The one or more openings can be positioned near the distal end of the valve member, but spaced proximal from the distal end by a distance. The valve member 2065 can have a tapered distal end or a step near the distal end, which can be configured to engage the housing 2010 (e.g., an inside of the male luer) to impede further movement of the valve member 2065 in the distal direction once it has reached the closed position. The distal end of the valve member 2064 can be substantially flush with the distal end of the housing 2010 (e.g., of the male luer).

When the valve member 2065 is displaced proximally, to the open position, the fluid path can extend through the distal opening (e.g., at the end of the male luer), through the one or more side openings of the valve member, into the channel of the valve member, out the proximal opening of the valve member, into a channel through the housing 2010 (e.g., through the third or proximal housing portion, and through the proximal opening (e.g., at the end of the female luer). Fluid could flow in either direction through the fluid flow path, depending on the situation. In some cases, fluid can enter a space between the exterior of the valve member 2065 and the inside of the male luer projection. A seal member 2067 (e.g., an O-ring) can form a seal between the exterior of the valve member 2065 and the housing 2010 (e.g., the first or distal housing portion), such as on an inside of the male luer projection. The seal member 2067 can impede the fluid from leaking out of the flow path 2008 through the connector 2002. The flow path 2008 can have a distal portion, which can be at least partially defined by the valve member 2065. The flow path 2008 can include a proximal portion, which can be at least partially defined by the housing 2010 (e.g., by the third or proximal housing portion 2010c), and by the female luer in some cases. A seal member 2069 (e.g., O-ring) can form a seal between the valve member 2065 and the housing 2010 (e.g., the third or proximal housing portion 2010c). The seal member 2069 can be on an exterior of the valve member 2065 and on an inside of the housing portion 2010c. The valve member 2065 can be received into the channel through the housing portion 2010c, which can be beneficial to allow the valve member 2064 to slide proximally without hitting the pawls 2025 on the housing portion 2010c. As the valve member 2065 moves to the open position, the shaft of the valve member 2065 can slide along the seal member 2067 and/or the seal member 2069. The one or more openings in the side of the valve member 2065 can remain distal of the seal member 2067 when the valve member 2065 is in the open position. The valve member 2065 can include a flange 2071 or one or more protrusions, which can extend laterally from the exterior of the valve member (e.g., from the valve member shaft).

A distal portion of the third housing portion 2010c can extend into the interior volume of the second housing portion 2010b. A proximal portion of the third housing portion 2010c can extend proximally out of the second housing portion 2010b, such as by a longitudinal distance of less than about 10 mm, less than about 9 mm, less than about 8 mm, less than about 7 mm, less than about 6 mm, less than about 5 mm, less than about 4 mm, less than about 3 mm, or less, or any ranges or values between any of these distances, although other configurations could be used. The proximal portion of the third housig portion 1020c can provide the femal luer, for example. The exterior of the proximal portion of the third housing portion 2010c, which extends outside of the second housing portion 2010b, can have a shaft and external threads or protrusions to engage corresponding threads for a luer lock connection. In some cases, the shaft does not include any other protrusions or steps other than the external threads or protrusions configured to engage corresponding threads for a luer lock connection. In some cases, the proximal portion of the third housing portion 2010c, which extends outside of the second housing portion 2010b does not have any protrusions or other features that extend laterally beyond the threads or luer lock protrusion(s). When another device is connected to the proximal end of the connector (e.g., to the female luer), the external threads (or other thread-engagement protrusion(s)) can be received into the other device so that they are not exposed. In some cases, the third housing portion 2010c does not have any protrusions, steps, recesses, or other external features that are exposed when the other device is connected to the proximal end of the connector. The connector 2002 can be configured so that when a standard male luer lock connector is coupled to the femal luer lock connection at the proximal end of the connector 2002, the housing of the other connector (e.g., the shroud of the male luer lock connector) can be spaced away from the proximal end of the second housing portion 2010b by a distance of less than about 10 mm, less than about 9 mm, less than about 8 mm, less than about 7 mm, less than about 6 mm, less than about 5 mm, less than about 4 mm, less than about 3 mm, less than about 2 mm, less than about 1 mm, or less, or any ranges or values between any of these distances, although other configurations could be used. Having only a small portion of the third housing portion 2010c exposed (e.g., with a gap of the distances discussed above), can prevent or impede the third housing portion 2010c from being gripped to loosen or remove the other device. The proximal portion of the valve member 2065 can extend into the distal portion of the third housing portion 2010c. A first or distal portion of the fluid pathway 2008 can extend through the valve member, and a second or proximal portion of the fluid pathway 2008 can extend through the third housing portion 2010c (e.g., the proximal end portion or female luer fitting). As the valve member 2065 moves toward the open position, more of the valve member 2065 can move into the third housing portion 2010c. A line (e.g., extending laterally perpendicular to a longitudinal axis of the connector 2002) can pass through the second housing portion 2010b, the third housing portion 2010c, and the valve member 2065. The valve member 2065 can be partially nested into the third housing portion 2010c, and the third housing portion 2010c can be partially nested into the second housing portion 2010b.

The connector 2002 can include one or more actuator arms 2073, which can be used to actuate. the valve member 2065 to the open position, as discussed herein. The actuator arms 2073 can extend distally from a base 2075. The base 2075 can have an opening, and the valve member 2065 can extend through the opening, until the flange 2071 abuts against the proximal side of the base 2075. The actuator arms 2073 can extend distally (e.g., through one or more opening or slots through the housing 2010) so that the arms 2073 as positioned in the space between the male luer projection and the shroud. The connector 2002 can include two arms 2073, which can be positioned opposite each other as show, although various other configurations or numbers of arms 2073 could be used.

As another connector (e.g., sometimes referred to as a mating connector) is coupled to the distal end 2004 of the connector 2002, the other (e.g., mating) connector can push the valve member 2065 proximally. In some cases, a projection on the mating connector can abut against the distal end of the valve member 2065 and can directly push the valve member 2065 in the proximal direction toward the open position. In some cases, during connection, the mating connector can push the one or more arms 2073 proximal. The base 2075 can push the flange 2071 distally, which can move the valve member 2065 toward the open configuration. In some embodiments, the arm(s) 2073 can be integrally formed with the valve member 2065. For example, the base 2075 can be a flange that extends laterally outward from the exterior of the valve member 2065.

In some embodiments, the arms 2073 can be coupled to the valve member 2065. For example, the opening through the base 2075 can have a friction fit with the valve member 2075. In some cases, the opening can be sized to have the friction fitting with the valve member 2065. With reference to FIG. 62, in some cases, the base 2075 can include one or more flexible tabs 2077, which can be deformed by the valve member 2065 as the valve member 2065 is inserted through the opening in the base 2075. The width of the opening with the tab(s) 2077 can be smaller than the width of the valve member 2065. The width of the opening without the tab(s) 2077 can be equal to or larger than the width of the valve member 2065. In some embodiments, the arm(s) 2073 can be coupled to the valve member 2065 by a keyed engagement, by snap fit elements, sonic welding, adhesive, screw threads, pins, or any other suitable coupling mechanism.

The valve member 2065 can be biased toward the closed position. The connector 2002 can include a biasing member 2079, which can bias the closure system 2006 to the closed configuration, such as by biasing the valve member 2065 to the distal or closed position. The biasing member 2079 can include a first securing portion or ring 2081 and a second securing portion or ring 2083. The first securing ring 2081 can be seated in a groove 2087 on the housing 2010 (e.g., on the first or distal housing portion 2010a), such as near the distal end. The second securing ring 2083 can be seated on the valve member 2065, such as proximal of the flange 2071. The biasing member 2079 can include one or more resilient portions or straps 2085, which can extend between the first ring 2081 and the second ring 2083. When the valve member 2065 is moved proximally, the flange 2071 can push the second securing ring 2083 proximally, while the first securing ring 2081 remains in the groove 2087, so that the resilient strap(s) 2085 stretch. The resilient band(s) 2085 can provide the biasing force to urge the valve member 2065 distally, such as back to the closed position. The second securing ring 2083 can press the flange 2061 in the distal direction, and the flange 2061 can press the base 2075 and/or the arms 2073 distally, in some implementations. The housing 2010 (e.g., the distal housing portion 2010a) can have one or more slot(s) 2089, which can receive the strap(s) 2085. The second housing portion 2010b can have one or more openings 2091, so that the strap(s) 2085 can extend from outside the housing 2010 to the interior of the housing 2010. The first securing portion or ring 2081 and/or the second securing portion or ring 2083 can be secured to the housing 2010 and/or to the valve 2065 in various other manners, such as using an adhesive, a clamp, a friction fitting, a bolt, a pin, or any other suitable coupling mechanism. The first securing portion or ring 2081 can be coupled on an exterior of the housing 2010 and the second securing portion or ring 2083 can be disposed inside an interior of the housing 2010.

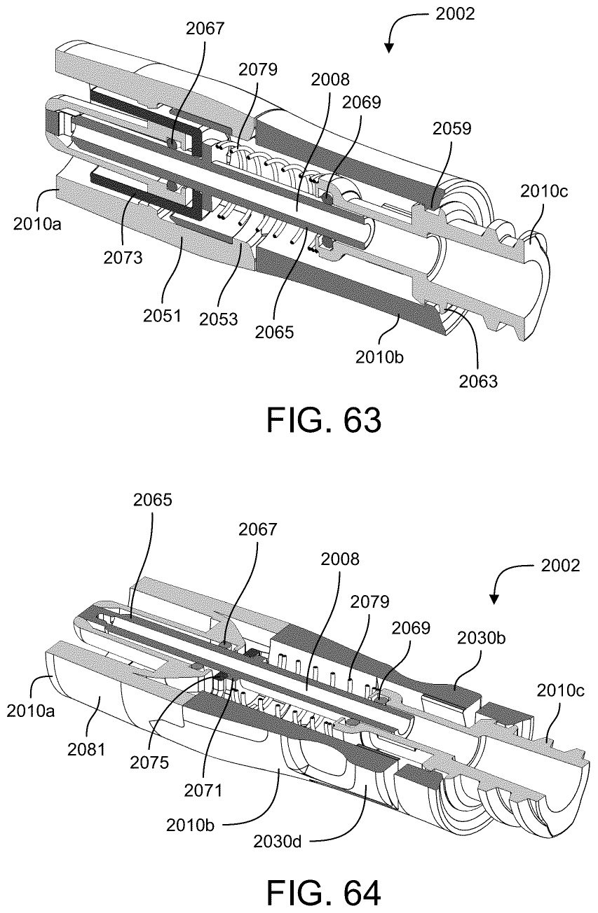

Various other biasing members could be used. FIG. 63 is a cross-sectional view of an example embodiment of a connector 2002 in which the biasing member 2079 is a spring. FIG. 64 is another cross-sectional view of the example connector 2002 with the spring biasing member 2079. The connector 2002 of FIGS. 63-64 can have features similar to the connector 2002 of FIGS. 55-62, or of other embodiments disclosed herein. The spring biasing member 2079 can have a first or distal end that can be seated against the flange 2071 (e.g., the proximal side thereof) of the valve member 2065. The spring biasing member 2079 can have a second or proximal end that can be seated against the third or proximal housing portion 2010c (e.g., the proximal end portion). Various alternative designs could be used. The spring biasing member 2079 can be seated against a portion of the second housing portion 2010b, or the base 2075 of the arm(s) could be disposed proximal of the flange 2071 and the spring 2079 can be seated against the base 2075, or against another component that is coupled to the valve member 2065. The connector 2020 with the spring biasing member 2079 can omit the opening(s) 2091, so that the housing 2010 can better impede foreign objects or contaminants from entering the interior of the housing 2010. Various other suitable biasing members could be used.

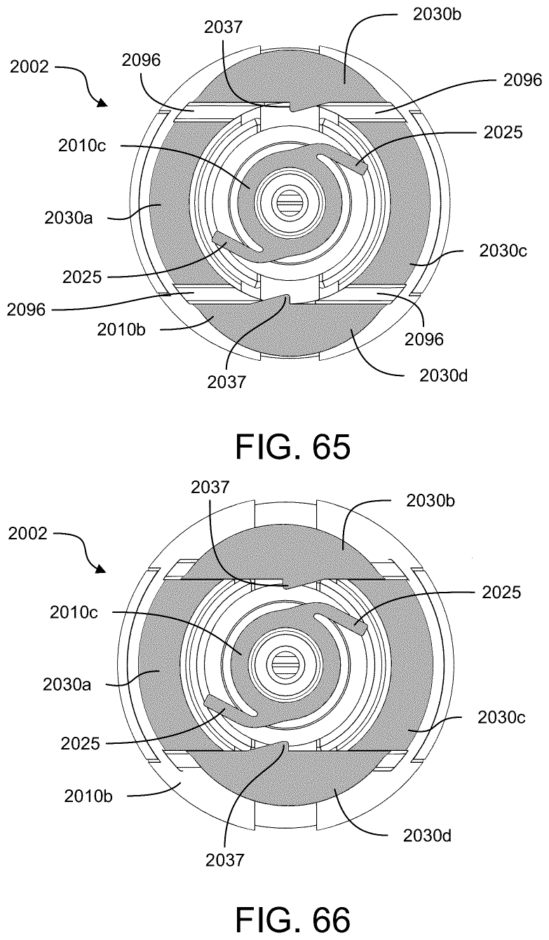

The connector 2002 can include a rotation mechanism 2012, which can include features similar to the other embodiments disclosed herein. The rotation mechanism 2012 can be configured to enable the third or proximal housing portion 2010c (e.g., including the proximal end 2014) to rotate relative to the second or central housing portion 2010b and/or relative to the first or distal housing portion 2010a (e.g., including the distal end 2004 or femail luer). In some embodiments, the rotation mechanism 2012 can be configured to permit rotation of the proximal end 2014 in a first direction (e.g., counterclockwise) and to impede rotation of the proximal end 2014 in a second direction (e.g., clockwise). FIG. 65 shows a cross-sectional view through the rotation mechanism 2012 of the connector 2002.

The housing 2010 (e.g., the second or center housing portion 2010b) can include multiple sidewall sections 2030a-d separated by gaps 2096 (e.g., similar to any of FIGS. 27-35, and 44). For example, the housing 2010 can have four sidewall sections 2030a-d. Opposing sidewall sections 2030b and 2030d can be pressed inwardly to transition the connector from a disengaged configuration or state to an engaged configuration or state. When released, the opposing sidewall sections 2030b and 2030d can return to the unengaged state. FIG. 65 shows the unengaged state. FIG. 66 shows the engaged state.

The opposing sidewall sections 2030b and 2030d can have one or more protrusions 2037, which can extend inwardly from the sidewall sections 2030b and 2030d. The protrusions 2037 can be similar to the housing teeth 2036 disclosed herein. In some embodiments, the projections 2037 can include an engagement surface, which can be similar to other embodiments disclosed herein. A line normal to the engagement surface can extend substantially tangential to a curve along the path of motion of the protrusions 2037, or varying from the tangential direction by about 20 degrees, about 15 degrees, about 10 degrees, about 5 degrees, about 3 degrees, about 2 degrees, about 1 degree, or less, or any ranges or values therebetween, although other configurations could be used.

The housing portion 2010c can include one or more pawls 2025, which can be configured to engage the protrusions 2037 when the connector is in the engaged state (e.g., with the sidewall sections 2030b and 2030d pressed inward). The pawls 2025 can be similar to other embodiments disclosed herein. The pawls 2025 can extend out from a connection point on the housing portion 2010c (e.g., from an exterior of the lumen forming part of the flow path 2008. The pawl 2025 can be curved so that an end portion of the pawl 2025 extends along a direction that is generally tangential to a curve along the path of motion of the rotating pawls 2025. The pawls 2025 can have an engagement surface and a sliding surface, similar to other embodiments discussed herein. A line normal to the engagement surface can extend generally tangentially to a curve along the path of motion of the engagement surface as the pawls 2025 rotate relative to the housing 2010, or varying from the tangential direction by about 20 degrees, about 15 degrees, about 10 degrees, about 5 degrees, about 3 degrees, about 2 degrees, about 1 degree, or less, or any ranges or values therebetween, although other configurations could be used.

When the connector 2002 is in the engaged configuration, rotation of the third housing portion 2010c in the tightening direction (e.g., clockwise in FIG. 66) relative to the second housing portion 2010b can cause the engagement surface(s) of the one or more pawls 2025 to abut against the engagement surface(s) of the one or more protrusions 2037 to impede further rotation of the third housing portion 2010c in the tightening direction relative to the second housing portion 2010b. In that configuration, further rotation of the third housing portion 2010c would cause the second housing portion 2010b to rotate with the third housing portion 2010c. This configuration can enable a user to attach another connector to the proximal end portion 2014 by twisting the other connector in the tightening direction while holding the housing 2010, as discussed herein.

When the connector 2002 is in the engaged configuration, rotation of the third housing portion 2010c in the loosening direction (e.g., counter clockwise in Figure) relative to the second housing portion 2010b can cause the pawl(s) 2025 to contact the protrusion(s) so that the pawl(s) 2025 are deflected to permit the pawl(s) 2025 to rotate past the corresponding protrusion(s) 2037. For example, the pawl 2025 can have a sliding surface that is configured to contact the protrusion 2037 and slide along the protrusion 2037 as the proximal end portion 2014 rotates in the loosening direction. The pawl 2025 can be deflected (e.g., inwardly) when the sliding surface of the pawl 2025 engages the protrusion 2037. When the pawl 2025 rotates past the protrusion 2037, the pawl 2025 can return to its default or unflexed position (e.g., shown in FIG. 65). The force that causes the one or more pawls 2025 to deform and rotate past the one or more protrusions 2037 can be less than the force to detach another connector that is coupled to the proximal end portion 2014 (e.g., to the female luer). Thus, if a user holds the first housing portion 2010a or the second housing portion 2010b and rotates the other connector in the loosening direction, the third housing portion 2010c will merely rotate with the other connector in the loosening direction relative to the second housing portion 2010b, thereby impeding disconnection of the other connector from the proximal end portion 2014. Thus, the connector 2002 can be configured to impede decoupling of another connector from the proximal end portion even when the connector 2002 is in the engaged configuration.

The distance that the sidewall sections 2030b and 2030d can be pressed inward can be limited by stops, similar to other embodiments disclosed herein. For example, when the second sidewall section 2030b is pressed inward, a first end of the second sidewall section 2030b can abut against a first end of the first sidewall section 2030a and/or a second end of the second sidewall section 2030b can abut against a first end of the third sidewall section 2030c. When the fourth sidewall section 2030d is pressed inward, a first end of the fourth sidewall section 2030d can abut against a second end of the first sidewall section 2030a and/or a second end of the fourth sidewall section 2030d can abut against a second end of the third sidewall section 2030c. Displacing the one or more sidewall sections 2030a-d to the engaged configuration can collapse one or more corresponding gaps 2096. The size of the corresponding gaps 2096 can set the displacement distance that the one or more sidewall sections 2030a-d can be displaced. When the one or more corresponding gaps 2096 are collapsed, the one or more sidewall sections 2030a-d can be impeded from being displaced further inward (e.g., even if additional squeezing force were applied).

The stops can impede the protrusions 2037 from being displaced so far inward that they would block the pawls 2025 and third housing portion 2010c from rotating in the loosening direction relative to the second housing portion 2010b. For example, when the sidewall sections 2030b and 2030d are pressed inward until they abut against the other sidewall sections 2030a and 2030c, the protrusions 2037 can be positioned so that they can displace the pawls 2025 inward to permit the proximal end portion 2014 to rotate in the loosening direction (e.g., counter clockwise in FIG. 66), and so that they can engage the pawls 2025 to impede rotation of the proximal end portion 2014 in the tightening direction (e.g., clockwise in FIG. 66).

When the connector 2002 is in the disengaged configuration (e.g., as shown in FIG. 66), the one or more protrusion(s) 2037 can be positioned to not engage, or to not contact, the pawls 2025 as the third housing portion 2020c rotates relative to the second housing portion 2010b. In the disengaged state, the connector 2002 can permit the third housing portion 2010c (e.g., the female luer connection or proximal end 2014) to spin freely in both directions relative to the second housing portion 2010b. This feature can be beneficial to avoid twisting or tangling a fluid line coupled to the connector 2002. This feature can be beneficial to avoid unintended disconnection of the connector 2002 from another connector. A threshold force to rotate the third housing portion 2010c in the loosening direction can be lower when the connector 2002 is in the disengaged state than when the connector 2002 is in the engaged state, even though both states can permit rotation of the third housing portion 2010c in the loosening direction relative to the housing. This can be because the pawls 2025 do not contact the protrusions 2037 when the third housing portion 2010c rotates relative to the second housing portion 2010b with the connector 2002 in the disengaged state, whereas the pawls 2025 are deflected by the protrusions 2037 to permit rotation of the third housing portion 2010c in the loosening direction when the connector 2002 is in the engaged state. In some embodiments, the rotation mechanism 2012 can have a free-spin configuration that enables the third housing portion 2010c to rotate in both directions relative to the second housing portion 2010b and/or distal end 2004 (e.g., when in the disengaged configuration).

The connector 2002 can include one or more buttons (e.g., the side wall sections 2030b and 2030d), which can be pressed inward by a user to transition the connector 2002 from the disengaged state to the engaged state. When the user is connecting a main ting connector to the femail luer, the user can press the button(s) inward to and can start to thread the mating connector onto the female luer, which can cause the female luer (and the rest of the third housing portion 2010c) to rotate in the tightening direction (e.g., clockwise). The pawls 2025 can engage the protrusions 2037 to impede further rotation of the third housing portion 2010c relative to the second housing portion 2010b. The user can then continue to thread the mating connector into engagement with the female luer (e.g., while holding the second housing portion 2010b and while pressing the button(s)). The user can release the button(s) (e.g., 2030b and 2030d), and the connector 2002 can transition to the disengaged state, which can permit the third housing portion 2010c (and the female luer) to rotate freely in both directions relative to the second housing portion 2010b, to impeded disconnection of the mating connector from the female luer (e.g., even if the user were to rotate the mating connector in the loosening direction while holding the second housing portion 2010b). The connector 2002 can transition between the engaged state and the disengaged state without axial movement of any component(s) of the connector 2002. The connector 2002 can transition between the engaged state and the disengaged state by radial movement of a portion of the housing 2010 (e.g., of the second housig portion 2010b). The connector 2002 can transition between the engaged state and the disengaged state by deformation of a portion of the housing 2010 (e.g., of one or both of sidewall portions 2030b and 2030d). The features that engage to impede rotation in the tightening direction (e.g., the pawls 2025 and the protrusions 2037) can be disposed inside the connector 2002, such as in an internal chamber defined by the second housing portion 2010b.

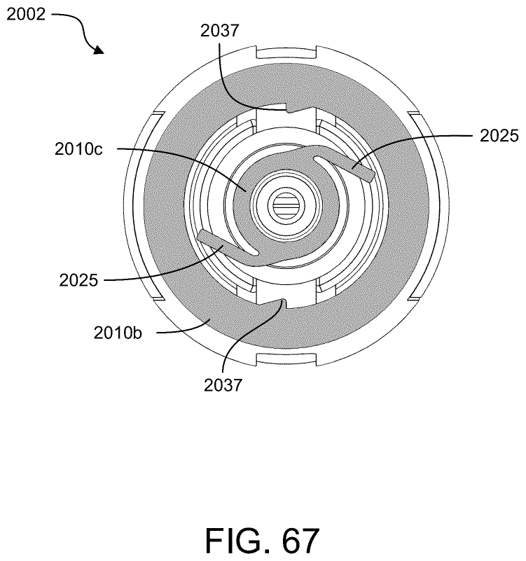

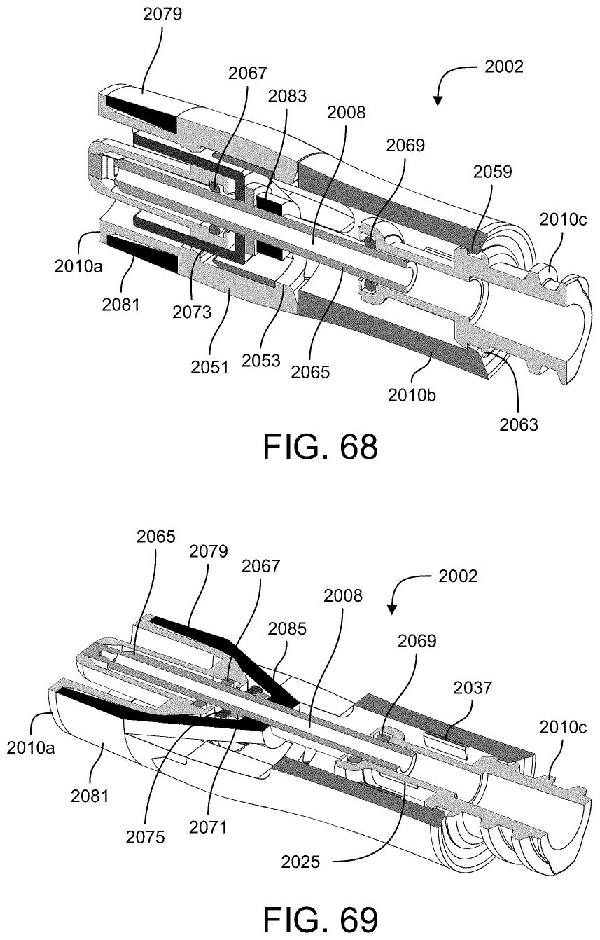

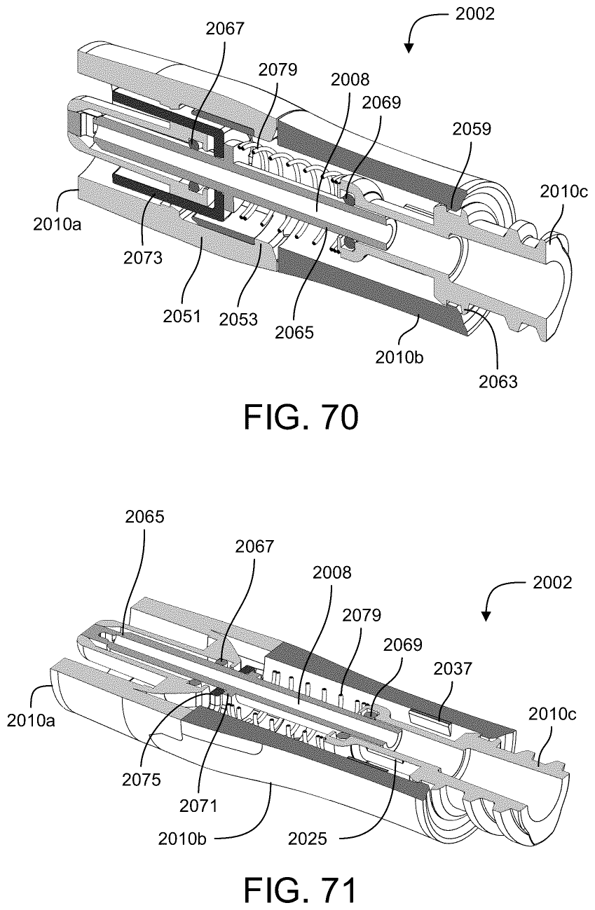

FIG. 67 shows a cross-section of an example embodiment of a connector 2002 with a rotation mechanism 2012 with a single state, which can be similar to the engaged state in the embodiment of FIG. 66. The rotation mechanism 2012 can be configured to permit rotation of the proximal end 2014 in a first direction (e.g., counterclockwise) and to impede rotation of the proximal end 2014 in a second direction (e.g., clockwise). The user does not need to hold down any buttons while coupling the mating connector to the female luer.

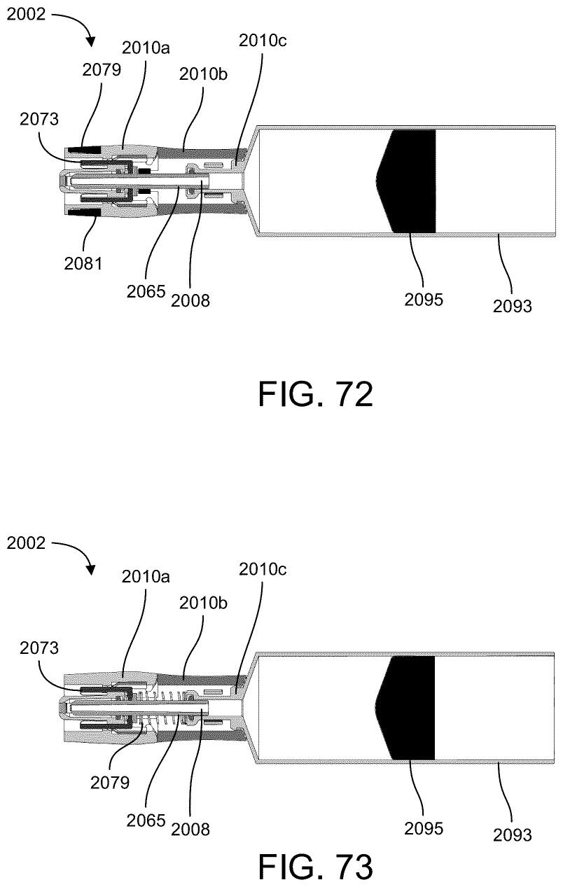

In some embodiments, a connector 2020 similar to those disclosed in other embodiments herein can be integrated into another medical device, such as a syring, a vial adapter, or a bag spike, etc. The third housing portion 2010c can an integral part of the other medical device, for example. FIG. 72 shows a connector 2002 coupled to a body of a syring 2093. The syringe can have a plunger 2095. The syring body 2093 can be integrally formed with, or coupled to, the third housing portion 2010c. In FIG. 72, the connector 2002 uses a strap biasing member 2079. FIG. 73 shows a similar connector coupled to a syring body 2093, where the connector 2002 has a spring biasing member 2079. In some embodiments, the connectors 2002 of FIGS. 72 and/or 73 can include buttons 2030b, 2030d or other actuators to transition the connector 2002 from the disengaged state to the engaged state. In some embodiments, the connector can have a single state, as discussed herein.

The housing 2010 (e.g., housing portions 2010a-c), the valve member 2065, the and the actuation arm(s) 2073 can be made of a substantially rigid material, such as polycarbonate plastic, although various other polymers and other materials could be used. The strap biasing member 2079, and the seal members 2069 and 2067 can be made of a resilient or flexible material, such as silicone, or rubber, or the like. The connector 2002 can have a biasing member 2079 for biasing the valve 2065 toward the closed position, and that biasing member can be independent of the structure that transitions the connector 2002 between the engaged state and the disengaged state. For example, when the straps 2085 are stretched, or when the spring is compressed, that does not apply force to the housing portions 2030b and 2030d that move to transition between the engaged and disengaged states. When the housing portions 2030b and 2030d move between the engaged and disengaged states, that does not apply force to the biasing member 2079 or to the valve member 2065.

Various alternatives and combinations of the disclosed features can be used. Also, the proportions and ratios of the sizes of various components, edges, and surfaces that are shown in the Figures are intended to form part of this disclosure, even when not specifically discussed.

Unless the context clearly requires otherwise, throughout the description and the claims, the words “comprise,” “comprising,” “include,” “including,” and the like are to be construed in an inclusive sense, as opposed to an exclusive or exhaustive sense; that is to say, in the sense of “including, but not limited to.” The words “coupled” or connected,” as generally used herein, refer to two or more elements that can be either directly connected, or connected by way of one or more intermediate elements. Additionally, the words “herein,” “above,” “below,” and words of similar import, when used in this application, shall refer to this application as a whole and not to any particular portions of this application. Where the context permits, words in the Detailed Description using the singular or plural number can also include the plural or singular number, respectively. The words “or” in reference to a list of two or more items, is intended to cover all of the following interpretations of the word: any of the items in the list, all of the items in the list, and any combination of the items in the list. All numerical values provided herein are intended to include similar values within a range of measurement error.

Although this disclosure contains certain embodiments and examples, it will be understood by those skilled in the art that the scope extends beyond the specifically disclosed embodiments to other alternative embodiments and/or uses and obvious modifications and equivalents thereof. In addition, while several variations of the embodiments have been shown and described in detail, other modifications will be readily apparent to those of skill in the art based upon this disclosure. It is also contemplated that various combinations or sub-combinations of the specific features and aspects of the embodiments may be made and still fall within the scope of this disclosure. It should be understood that various features and aspects of the disclosed embodiments can be combined with, or substituted for, one another in order to form varying modes of the embodiments. Any methods disclosed herein need not be performed in the order recited. Thus, it is intended that the scope should not be limited by the particular embodiments described above.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. Any headings used herein are for the convenience of the reader only and are not meant to limit the scope.

Further, while the devices, systems, and methods described herein may be susceptible to various modifications and alternative forms, specific examples thereof have been shown in the drawings and are herein described in detail. It should be understood, however, that the disclosure is not to be limited to the particular forms or methods disclosed, but, to the contrary, this disclosure covers all modifications, equivalents, and alternatives falling within the spirit and scope of the various implementations described. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including ambient temperature and pressure.

1. A medical connector comprising:
a housing comprising first, second, third, and fourth sidewall sections, wherein the second and fourth sidewall sections comprise protrusions that extend inwardly, wherein the second and fourth sidewall sections are configured to be pressed inwardly towards each other to transition the medical connector from a disengaged state to an engaged state when an external force is applied, and wherein the second and fourth sidewall section are configured to automatically move outwardly away from each other to transition the medical connector from the engaged state to the disengaged state when the external force is removed; and
a proximal end portion comprising a connection fitting configured to couple to another connector by rotation of the other connector relative to the proximal end portion in a tightening direction, the proximal end portion comprising protrusions configured to engage the protrusions on the second and fourth sidewall sections of the housing when the medical connector is in the engaged state to impede rotation of the proximal end portion relative to the housing in the tightening direction to facilitate coupling of the other connector to the proximal end portion, wherein the protrusions on the proximal end portion are configured to not engage the protrusions on the second and fourth sidewall sections of the housing when the medical connector is in the disengaged state, so that the proximal end portion rotates relative to the housing in the tightening direction and in a loosening direction opposite the tightening direction.

2. The medical connector of claim 1, configured to permit rotation of the proximal end portion relative to the housing in the loosening direction when the medical connector is in the engaged state to impede disconnection of the other connector from the proximal end portion.

3. The medical connector of claim 1, wherein the protrusions on the proximal end portion and/or the protrusions on the housing are configured to deform to permit the proximal end portion to rotate relative to the housing in the loosening direction when the medical connector is in the engaged state.

4. The medical connector of claim 1, wherein either the protrusions on the proximal end portion or the protrusions on the housing includes pawls that are configured to flex to permit the protrusions on the proximal end portion to rotate past the protrusions on the distal end portion in the loosening direction when the medical connector is in the engaged state.

5. The medical connector of claim 1, configured such that when the medical connector is in the engaged state, rotating the proximal end portion relative to the housing in the loosening direction by a force that is below a disconnection threshold for loosening or removing the attached other connector from the proximal end portion causes the protrusions on the proximal end portion to rotate past the protrusions on the housing, to thereby impede disconnection of the other connector from the proximal end portion.

6. The medical connector of claim 1, wherein the protrusions on the proximal end portion comprise flexible pawls.

7. The medical connector of claim 1, wherein the protrusions on the second the fourth sidewall sections of the housing comprise flexible pawls.

8. The medical connector of claim 1, wherein the first and third sidewall sections define stop surfaces that limit the distance that the second and fourth sidewall sections can be pressed inwardly towards each other.

9. The medical connector of claim 1, wherein the first and third sidewall sections are positioned to abut against the second and fourth sidewall sections when the medical connector is in the engaged state, to limit engagement between the protrusions so that the proximal end portion can rotate in the loosening direction relative to the housing.

10. The medical connector of claim 1, wherein the connection fitting on the proximal end portion is a luer lock fitting.

11. The medical connector of claim 1, wherein the connection fitting on the proximal end portion is a female luer lock fitting.

12. The medical connector of claim 1, wherein the proximal end portion does not move axially relative to the housing.

13. The medical connector of claim 1, wherein gaps separate the first, second, third, and fourth sidewall sections.

14. The medical connector of claim 1, wherein the protrusions on the housing are configured to directly contact the protrusions on the proximal end portion in the engaged state.

15. The medical connector of claim 1, comprising an engagement member that has one or more inner engagement elements that are configured to interface with the protrusions on the proximal end portion, wherein the engagement member has one or more outer engagement elements that are configured to interface with the protrusions on the housing.

16. The medical connector of claim 1, wherein the proximal end portion includes a first opening, wherein the housing comprises a second opening, and wherein the medical connector includes a fluid pathway between the first opening and the second opening.

17. The medical connector of claim 1, comprising a valve member configured to move between a closed position that closes the fluid pathway and an open position that opens the fluid pathway, wherein the valve member includes a fluid channel, wherein a proximal end of the valve member extend into the proximal end portion.

18. The medical connector of claim 17, comprising a seal member configured to provide a fluid seal between an exterior of the valve member and an interior of the proximal end portion.

19. The medical connector of claim 1, further comprising a biasing member configured to bias the valve member toward the cloased position.

20. The medical connector of claim 19, wherein the biasing member comprises:
a first securing portion coupled to the housing;
a second securing portion coupled to the valve member; and
one or more resilient straps that couple the first securing portion to the second securing portion, wherein the second securing portion moves away from the first securing portion as the valve member moves from the closed position toward the open position, thereby stretching the one or more resilient straps.

21. The medical connector of claim 19, wherein the biasing member comprises a spring.

22.-78. (canceled)
